(19) United States
(12) Patent Application Publication
(10) Pub. No.: US 2024/0279191 A1
(43) Pub. Date: Aug. 22, 2024

(54) SMALL MOLECULE INHIBITORS OF TEAD-YAP

(71) Applicant: The General Hospital Corporation, Boston, MA (US)

(72) Inventors: Xu Wu, Lexington, MA (US); Lu Hu, Malden, MA (US)

(21) Appl. No.: 18/694,815

(22) PCT Filed: Sep. 22, 2022
(86) PCT No.: PCT/US2022/076868

§ 371 Date: Mar. 22, 2024

Related U.S. Application Data
(60) Provisional application No. 63/248,047, filed on Sep. 24, 2021.

(51) Int. Cl. C07D 295/192; A61P 35/00; C07D 211/22; C07D 213/30; C07D 213/75; C07D 239/42; C07D 249/06; C07D 295/26; C07D 305/08; C07D 309/06

(52) U.S. Cl. CPC C07D 295/192; A61P 35/00; C07D 211/22; C07D 213/30; C07D 213/75; C07D 239/42; C07D 249/06; C07D 295/26; C07D 305/08; C07D 309/06

(57) ABSTRACT

The present disclosure relates to compounds of Formula (I), and pharmaceutically acceptable salts thereof, compositions comprising same, and methods of using the compounds and compositions to treat various cancers.

### CROSS-REFERENCE TO RELATED APPLICATIONS

This application claims the benefit of U.S. Provisional Application No. 63/248,047, filed on Sep. 24, 2021, the entire disclosure of which is incorporated herein by reference in its entirety.

### STATEMENT REGARDING FEDERALLY SPONSORED RESEARCH

This invention was made with government support under grant numbers R01CA219814 and R01CA238270. The government has certain rights in the invention.

### FIELD

The present disclosure relates to the fields of chemistry and biology. More specifically, to compounds of Formula (I), and pharmaceutically acceptable salts thereof, as well as compositions and methods of using same to treat cancer.

### BACKGROUND

Hippo signaling is an emerging tumor suppressor pathway that plays key roles in normal physiology and tumorigenesis, through the regulation of cellular proliferation and survival. The signal transduction involves a core kinase cascade, including MST1/2 and Lats1/2 kinases, leading to YAP and TAZ phosphorylation, cytoplasmic retention and inhibition. YAP and TAZ are transcription co-activators, which bind to TEA-domain transcription factors (TEAD1-4 in mammals) and mediate transcriptional regulation. Upstream Hippo pathway components, such as SAV1, NF2/merlin, Mst1/2 and Lats1/2 are well characterized as tumor suppressors. Loss-of-function mutations or epigenetic silencing of these genes have been found in multiple human and mouse cancers. Consistently, liver specific knockout of NF2/merlin, or Mst1/2 causes hepatocellular carcinoma in mice.

The downstream transcription co-activator YAP/TAZ and TEAD1-4 are oncogenes amplified at high frequencies in many human and mouse tumors, including medulloblastoma, cutaneous squamous cell carcinoma, and cancers of lung, pancreas, esophagus, liver and colon. YAP/TAZ also confers resistance to standard chemotherapy, and high YAP activity correlates with poor prognosis of ovarian cancer patients. These results suggested that targeting YAP could be a good strategy for cancer therapeutics. Therefore, understanding how Hippo-YAP signaling is regulated would reveal novel cancer therapeutic targets for drug development. YAP/TAZ does not have DNA binding domain, therefore, its interaction with TEADs is essential to mediate YAP/TAZ oncogenic activities, and TEAD-YAP interaction has been proposed as a potential cancer therapeutic target. TEADs are also highly expressed in many cancers. However, the protein-protein interaction interface is shallow, and spanning a large surface area. Therefore, it has been very challenging to develop small molecule inhibitors of TEAD-YAP interaction. Moreover, most of the upstream druggable targets of Hippo pathway are tumor suppressor kinases, therefore, unsuitable for cancer drug development. Given the highly relevance of YAP-TEAD in multiple cancers (liver, pancreas, melanoma, colon, lung cancers), this is a highly competitive research area with huge potential for important cancer drug development.

### SUMMARY

Some embodiments provide a compound of Formula (I)

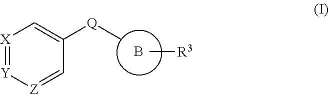

or a pharmaceutically acceptable salt thereof, wherein:
X is —CA-R¹, CH, N, or CR²;
Y is —CA-R¹, CH, N, or CR²;
Z is —CA-R¹, CH, N, or CR², wherein only one of X, Y, and Z is —CA-R¹;
Q is C(═O), S(═O), S(O₂), 4-5 membered spiroheterocyclyl, C1-C6 alkylene, or a bond;
A is O or NH;
R¹ is C1-C6 alkyl optionally substituted with:
(i) C3-C8 cycloalkyl optionally substituted with 1-2 substituents independently selected from halogen and C1-C6 alkyl,
(ii) 4-10 membered heterocyclyl optionally substituted with C1-C6 alkyl, acyl, or a C-linked ester,
(iii) 5-6 membered heteroaryl optionally substituted with C1-C6 alkyl or C1-C6 haloalkyl,
(iv) phenyl optionally substituted with C1-C6 alkyl or C1-C6 haloalkyl;
   each R² is independently halogen, cyano, C1-C6 alkyl, C1-C6 alkoxy, or C1-C6 haloalkyl;
Ring B is phenyl optionally substituted with 1-2 independently selected C1-C6 alkyl, 5-6 membered heteroaryl optionally substituted with 1-2 independently selected C1-C6 alkyl, or 4-10 membered heterocyclyl optionally substituted with 1-2 independently selected C1-C6 alkyl;
R³ is —C(═O)NR^{A}, —C(═O)OR^{A}, —NH(C═O)R^{A}, —NHC(═O)NH, or —C1-C6 alkyl(NHC(═O)NH)R^{A};
R^{A} is phenyl, 5-6 membered heterocyclyl, or 5-6 membered heteroaryl, each optionally substituted with 1-2 independently selected R^{A1};
each R^{A1} is independently —NR^{B}R^{C}, C-linked ester, —CO₂H, —S(O₂)NH₂, —NHC(═O)C1-C6 alkyl, or C1-C6 alkyl optionally substituted with hydroxyl, wherein
R^{B} and R^{C} is independently hydrogen, C1-C6 alkyl, C1-C6 haloalkyl, or
R^{B} and R^{C}, together with the nitrogen to which they are attached form a 4-6 membered heterocyclyl optionally substituted with 1-2 independently selected halogen, C1-C6 alkyl, C1-R⁶ haloalkyl, hydroxyl, or amino.

Some embodiments provide a composition comprising a compound of formula (I), or a pharmaceutically acceptable salt thereof and at least one pharmaceutically acceptable excipient.

Some embodiments provide a composition comprising a compound of formula (I), or a pharmaceutically acceptable salt thereof that is a small molecule inhibitor of TEAD-YAP.

Also provided herein is a method of treating cancer in a subject in need thereof, comprising administering to the subject an effective amount of a compound of formula (I), or a pharmaceutically acceptable salt thereof, or a pharmaceutical composition described herein.

### DESCRIPTION OF DRAWINGS

FIG. 1. Shows inhibition of TEAD2 auto-palmitoylation with treatment of TM2 and analogues under 0.05 and 0.5 μM for 30 mins, respectively.

FIG. 2. Shows identification of TM2 as novel TEAD auto-palmitoylation inhibitors.

FIG. 2A. IC₅₀ values for TM2 inhibition of TEAD2 auto-palmitoylation were characterized by western blot analysis (left) and quantified by Image J (right). FIG. 2B. IC₅₀ values for TM2 inhibition of TEAD4 auto-palmitoylation were characterized by western blot analysis (left) and quantified by Image J (right). FIG. 2C. The data was determined by independent replicates (n=3), and shown as mean±SEM. Palmitoylation of Myc-TEAD1 were analyzed by immunoprecipitation assay with treatment of TM2 at indicated concentrations for 24 h. FIG. 2D. The data was determined by independent replicates (n=3), and shown as mean±SEM. Palmitoylation of endogenous pan-TEAD (F) were analyzed by immunoprecipitation assay with treatment of TM2 at indicated concentrations for 24 h.

FIG. 3. Shows co-crystal structure of TEAD2 complexed with TM2. FIG. 3A. Ribbon diagram of the crystal structure of TEAD2-TM2. TM2 is shown in stick format. FIG. 3B. Close-up view of the TM2 binding site of TEAD2 with the superposition of the TEAD2-PLM structure (PDB 5HGU). Surrounding residues are shown in stick format. PLM is shown in stick format.

FIG. 3C. Conformational changes in side chains of residues in the new pocket in the presence of TM2 binding. Indicated residues from TEAD2-TM2 and TEAD2-PLM are shown in stick format. Distances (in Angstroms) between atoms are shown with dashed lines. FIG. 3D. Structural superposition of TEAD2-TM2, TEAD2-PLM (PDB 5HGU), and TEAD2-CP1 (PDB 6CDY). TEAD2 is shown as a ribbon. TM2, PLM (Palmitic acid), and CP1 are shown in stick format.

FIG. 4. Shows co-crystal structure of TEAD2 complexed with TM2. FIG. 4A. Comparison of orientations of TM2 and PLM in the binding pocket. The TEAD2 protein is shown in ribbon, with the pocket shown by surface. PLM and TM2 are shown in stick format. FIG. 4B. Structural superposition of TEAD2-TM2, TEAD2-PLM (PDB 5HGU), and TEAD3-VT105 (PDB 7CNL). TM2, PLM, and VT105 are shown in stick format. FIG. 4C. Structural superposition of TEAD2-TM2, TEAD2-PLM (PDB 5HGU), and TEAD1-K975 (PDB 7CMM). TM2, PLM, and K975 are shown in stick format. FIG. 4D. The Fo-Fc omit electron density map for TM2 at the contour level of 2.5 σ is shown in webbing. The TEAD2 protein is shown in ribbon and TM2 is shown in stick format.

FIG. 5. TM2 suppressed transcriptional outputs of Hippo pathway in cancer cells. FIG. 5A H226 cells were treated with TM2 at indicated concentrations for 24 h. The interactions of YAP and Pan-TEAD as well as TEAD1 was observed with YAP Co-IP. FIG. 5B. representative target genes of Hippo pathway in H226 cells were measured with treatment of TM2 at indicated concentrations for 48 h. The data was determined by independent triplicates and shown as mean SEM. FIG. 5C. Heatmap analysis of global genes transcriptional alteration in H226 treated with vehicle control or TM2. FIG. 5D. PCA biplot with genes plotted in two dimensions using their projections onto the first two principal components, and 4 samples (Control 2 samples, TM2 2 samples) plotted using their weights for the components. FIG. 5E. Gene set enrichment analysis of H226 cells treated with TM2 using oncogenic signature gene sets from Molecular Signatures Database. FIG. 5F. Gene set enrichment plot of Cordernonsi_YAP_conserved_Signature (left panel) and YAP_TAZ-TEAD Direct Target Genes (right panel) with H226 cells treated with TM2.

FIG. 6. Target gene expression in H226 with TM2 treatment for 24 h. The data was determined by independent triplicates and shown as mean±SEM.

FIG. 7. Heatmap analysis of YAP/TAZ-TEAD direct target genes transcriptional alteration in H226 xenograft tumor treated with K975 (GSE156912) and H226 cell line treated with TM2 (1 μM).

FIG. 8. TM2 showed inhibition on YAP dependent proliferation. FIG. 8A. Percentages of survival organoids with treatment of control or TM2 at indicated concentrations. FIG. 8B. Immunofluorescent staining of Ki67 in organoids treated with control or TM2 (40 nM). Pink, Ki-67; blue, nuclear DNA (DAPI). Bar, 20 μm. FIG. 8C. Cell inhibition in H226 cells with treatment of compounds at indicated concentrations for 6 days. The data was determined by independent triplicates and shown as mean±SEM. FIG. 8D. Cell inhibition in MSTO-211H, H2052, H28, HCT116 and DLD1 cells with treatment of TM2 at indicated concentrations for 5, 7, 6, 5, 5, 5 days, respectively. The data was determined by independent triplicates and shown as mean±SEM. FIG. 8E. Drug combination experiments using TM2 and MEK inhibitor Trametinib in DLD1: Heatmaps show color-coding as percentage of cell viability normalized to untreated controls. Heatmaps of Bliss score for TM2 and Trametinib combination were shown.

FIG. 9. Bright field images of organoids treated with control or TM2 (40 nM). Bar, 400 μm.

FIG. 10. Drug combination experiments using TM2 and MEK inhibitor Trametinib in HCT116: Heatmaps show color-coding as percentage of cell viability normalized to untreated controls. Heatmaps of Bliss score for TM2 and Trametinib combination were shown.

FIG. 11. Shows H226 cell proliferation data for TM2 analogues.

### DETAILED DESCRIPTION

TEADs have auto-palmitoylation activities: they may possess intrinsic “enzymatic activities” and utilize palmitoyl-CoA as a substrate. TEAD auto-palmitoylation inhibitors as chemical tools to study TEADs' functions were identify. A synthetic small molecule library was screened using a TEAD palmitoylation biochemical assay and identified more than 20 hits, which potently inhibit TEAD2 auto-palmitoylation. One compound (TM2) was focused on for further studies, and medicinal chemistry modifications. It was found that this compound binds to TEAD selectively. The crystal structure revealed that this compound (TM2) inserts into the deep hydrophobic pocket once occupied by palmitate. In addition, the head group of this compound occupies a new pocket located near TEAD-YAP binding interface and displaces TEAD side chains to push away YAP binding. Therefore, this compound is far more potent than any other known TEAD inhibitors in blocking TEAD-YAP activities. About 20 analogues of the TM2 compound was synthesized to explore the SAR of the core pharmacophore, aiming to improve its potency and selectivity, and to identify “lead” compounds for drug development. These compounds showed good potency in vitro and good correlation with target gene inhibition and cell-based activities in H226 cell proliferation studies. Also, these analogues have improved metabolic stability. The co-crystal structure shows TM2 occupies an additional unique pocket, ie, it binds to TEAD differently as compared to other inhibitors. Testing in mesothelioma cell lines with NF2 or Lats1 mutation shows that these cell lines are very sensitive to TM2. In addition, TM2 inhibits PDL1 expression in cancer cell lines, potentially serve as immune-modulating agents in cancers.

In one aspect the invention comprises small molecule inhibitors of TEAD-YAP. The small molecule inhibitors included in the invention are shown in the accompanying drawings. In another aspect the invention comprises a method of treating cancers by administering a small molecule inhibitor of the invention. The cancers include cancers of the liver, pancreas, melanoma, colon, and lungs.

### Compounds

Some embodiments provide a compound of Formula (I)

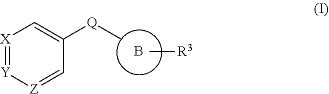

or a pharmaceutically acceptable salt thereof, wherein:
X is —CA-R¹, CH, N, or CR²;
Y is —CA-R¹, CH, N, or CR²;
Z is —CA-R¹, CH, N, or CR², wherein only one of X, Y, and Z is -A-R¹;
Q is C(═O), S(═O), S(O₂), 4-5 membered spiroheterocyclyl, C1-C6 alkylene, or a bond;
A is O or NH;
R¹ is C1-C6 alkyl optionally substituted with:
   (i) C3-C8 cycloalkyl optionally substituted with 1-2 substituents independently selected from halogen and C1-C6 alkyl,
   (ii) 4-10 membered heterocyclyl optionally substituted with C1-C6 alkyl, acyl, or a C-linked ester,
   (iii) 5-6 membered heteroaryl optionally substituted with C1-C6 alkyl or C1-C6 haloalkyl, or
   (iv) phenyl optionally substituted with C1-C6 alkyl or C1-C6 haloalkyl;
each R² is independently halogen, cyano, C1-C6 alkyl, C1-C6 alkoxy, or C1-C6 haloalkyl;
Ring B is phenyl optionally substituted with 1-2 independently selected C1-C6 alkyl, 5-6 membered heteroaryl optionally substituted with 1-2 independently selected C1-C6 alkyl, or 4-10 membered heterocyclyl optionally substituted with 1-2 independently selected C1-C6 alkyl;
R³ is —C(═O)NR^{A}, —C(═O)OR^{A}, —NH(C═O)R^{A}, —NHC(═O)NH^{A}, or —C1-C6 alkyl(NHC(═O)NH)R^{A};
R^{A} is phenyl, 5-6 membered heterocyclyl, or 5-6 membered heteroaryl, each optionally substituted with 1-2 independently selected R^{A1};
each R^{A1} is independently —NR^{B}R^{C}, C-linked ester, —CO₂H, —S(O₂)NH₂, —NHC(═O)C1-C6 alkyl, or C1-C6 alkyl optionally substituted with hydroxyl, wherein
R^{B} and R^{C} is independently hydrogen, C1-C6 alkyl, C1-C6 haloalkyl, or
R^{B} and R^{C}, together with the nitrogen to which they are attached form a 4-6 membered heterocyclyl optionally substituted with 1-2 independently selected halogen, C1-C6 alkyl, C1-R⁶ haloalkyl, hydroxyl, or amino.

In some embodiments, X is —CA-R¹. In some embodiments, Y is —CA-R¹. In some embodiments, Z is —CA-R¹.

In some embodiments, two of X, Y, and Z are both CH. In some embodiments, the partial structure of Formula (I) is

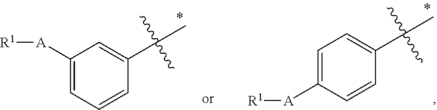

where the asterisk indicates the point attachment to Q.

In some embodiments, two of X, Y, and Z are both N. In some embodiments, the partial structure of Formula (I) is

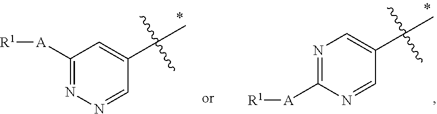

where the asterisk indicates the point attachment to Q.

In some embodiments, two of X, Y, and Z are both CR². In some embodiments, the partial structure of Formula (I) is

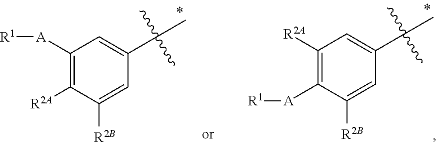

where the asterisk indicates the point attachment to Q and R^{2A} and R^{2B} are independently selected from R².

In some embodiments, two of X, Y, and Z is independently CH or N; wherein one of X, Y, and Z is CH. In some embodiments, the partial structure of Formula (I) is N

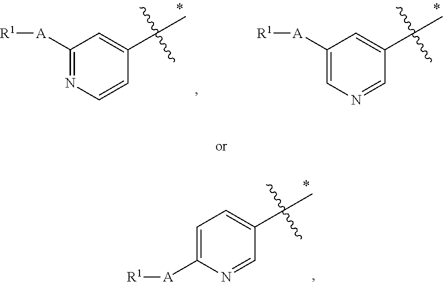

where the asterisk indicates the point attachment to Q.

In some embodiments, two of X, Y, and Z is independently CH or CR²; wherein one of X, Y, and Z is CH. In some embodiments, the partial structure of Formula (I) is

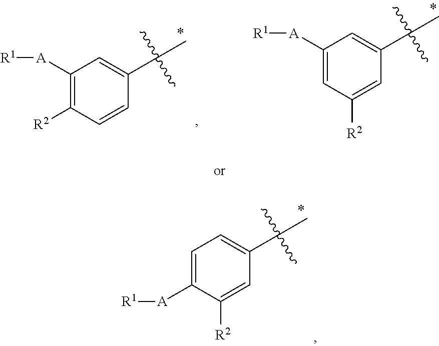

where the asterisk indicates the point attachment to Q.

In some embodiments, two of X, Y, and Z is independently N or CR²; wherein one of X, Y, and Z is N. In some embodiments, the partial structure of Formula (I) is

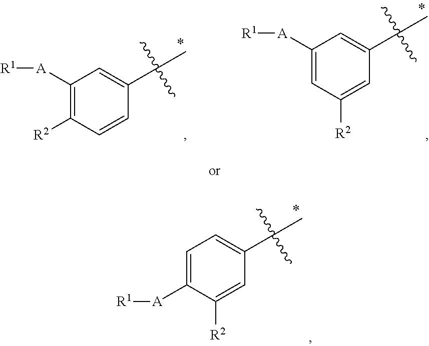

where the asterisk indicates the point attachment to Q.

In some embodiments, Q is C(═O). In some embodiments, Q is S(═O). In some embodiments, Q is S(O₂). In some embodiments, Q is 4-5 membered spiroheterocyclyl. In some embodiments, Q is a 4-membered spiroheterocyclyl. In some embodiments, Q is a 5-membered spiroheterocyclyl. In some embodiments, the spiroheterocyclyl is selected from oxetanyl, thietanyl, azetidinyl, tetrahydrofuranyl, tetrahydrothiophenyl, pyrrolidinyl, isoxazolidinyl, isothiazolidinyl, pyrazolidinyl, pyrrolidinonyl, and dihydrofuranonyl. In some embodiments, Q is

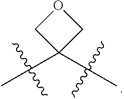 [SMILES: *C1(*)COC1]

In some embodiments Q is C1-C6 alkylene. In some embodiments, Q is C1-C3 alkylene.

In some embodiments, Q is methylene.

In some embodiments, Q is a bond.

In some embodiments, wherein A is O.

In some embodiments, A is NH.

In some embodiments, R¹ is C1-C6 alkyl optionally substituted with C3-C8 cycloalkyl optionally substituted with 1-2 substituents independently selected from halogen and C1-C6 alkyl. In some embodiments, R¹ is C1-C3 alkyl optionally substituted with C3-C6 cycloalkyl optionally substituted with 1-2 substituents independently selected from halogen and C1-C6 alkyl. In some embodiments, R¹ is C1-C2 alkyl substituted with C3-C6 cycloalkyl optionally substituted with 1-2 substituents independently selected from halogen and C1-C6 alkyl. In some embodiments, R¹ is selected from the group consisting of

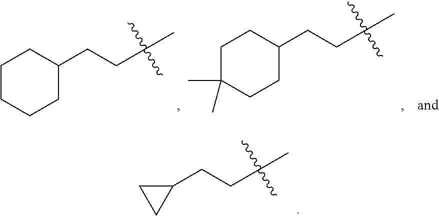 [SMILES: *CCC1CCCCC1 ; *CCC1CCC(C)(C)CC1 ; *CCC1CC1]

In some embodiments, R¹ is C1-C6 alkyl optionally substituted with 4-10 membered heterocyclyl optionally substituted with C1-C6 alkyl, acyl, or a C-linked ester. In some embodiments, R¹ is C1-C3 alkyl optionally substituted with 5-6 membered heterocyclyl optionally substituted with C1-C6 alkyl, acyl, or a C-linked ester. In some embodiments, R¹ is C1-C3 alkyl substituted with optionally substituted with 5-6 membered heterocyclyl optionally substituted with C1-C6 alkyl, acyl, or a C-linked ester. In some embodiments, R¹ is C1-C3 alkyl substituted with optionally substituted with 5-6 membered heterocyclyl selected from the group consisting of pyrrolidinyl, tetrahydrofuryl, thiolanyl, pyrazolinyl, oxathiolanyl, isoxazolidinyl, isothiazolidinyl, pyrrolinyl, pyrrolidinonyl, pyrazolidinyl, imidazolinyl, dioxolanyl, sulfolanyl, thiazolidedionyl, succinimidyl, dihydrofuranonyl, pyrazolidinonyl, oxazolidinyl, isoxazolidinonyl, hydantionyl, thiohydantionyl, imidazolidinonyl, oxazolidinonyl, thiazolidinonyl, oxathiolanonyl, dioxolanonyl, dioxazolidinonyl, oxadiazolidinonyl, triazolidinonyl, triazolidinethionyl, oxadiazolidinethionyl, dioxazolidinethionyl, dioxolanethionyl, oxazolidinethionyl, imidazolidinethionyl, isothiazolidinonyl, piperidinyl, tetrahydropyranyl, thianyl, morpholinyl, thiomorpholinyl, dioxanyl, piperazinyl, dithianyl, oxazinyl, tetrahydropyranonyl, piperidinonyl, dioxanonyl, oxazinanonyl, morpholinonyl, thiomorpholinonyl, piperazinonyl, tetrahydropyrimidinonyl, piperidinedionyl, oxazinanedionyl, dihydropyrimidindione, tetrahydropyridazinonyl, triazinanonyl, oxadiazinanonyl, dioxazinanonyl, morpholinedionyl, piperazinedionyl, piperazinetrionyl, and triazinanedionyl.

In some embodiments, R¹ is C1-C3 alkyl substituted with optionally substituted with 5-6 membered heterocyclyl selected from tetrahydropyranyl and piperidinyl. In some embodiments, R¹ is selected from the group consisting of

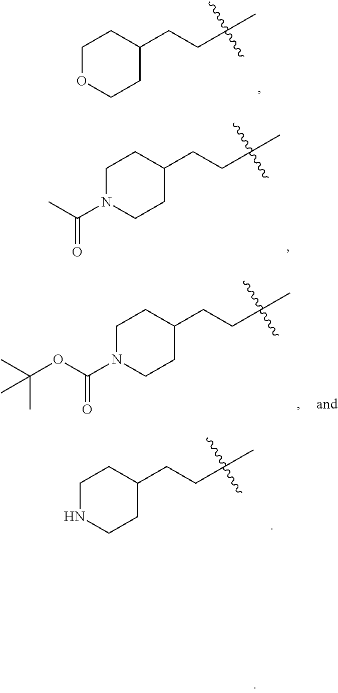 [SMILES: *CCC1CCOCC1 ; *CCC1CCN(C(C)=O)CC1 ; *CCC1CCN(C(=O)OC(C)(C)C)CC1 ; *CCC1CCNCC1]

In some embodiments, R¹ is C1-C6 alkyl optionally substituted with 5-6 membered heteroaryl optionally substituted with C1-C6 alkyl or C1-C6 haloalkyl. In some embodiments, R¹ is C1-C3 alkyl optionally substituted with 5-6 membered heteroaryl optionally substituted with C1-C6 alkyl or C1-C6 haloalkyl. In some embodiments, R¹ is C1-C3 alkyl substituted with 5-6 membered heteroaryl optionally substituted with C1-C6 alkyl or C1-C6 haloalkyl. In some embodiments, R¹ is C1-C3 alkyl substituted with optionally substituted 5-6 membered heteroaryl selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, thiatriazolyl, pyridinyl, pyrimidinyl, pyrazinyl, pyridazinyl, and triazinyl. In some embodiments, R¹ is selected from the group consisting of —CH₂CH₂(2-pyridyl), —CH₂CH₂(3-pyridyl), and —CH₂CH₂(4-pyridyl).

In some embodiments, R¹ is C1-C6 alkyl optionally substituted with phenyl which is optionally substituted with C1-C6 alkyl or C1-C6 haloalkyl. In some embodiments, R¹ is C1-C3 alkyl substituted with phenyl which is optionally substituted with C1-C6 alkyl or C1-C6 haloalkyl. In some embodiments, R¹ is C1-C3 alkyl substituted with phenyl optionally substituted with C1-C4 alkyl or C1-C3 haloalkyl. In some embodiments, R¹ is selected from the group consisting of

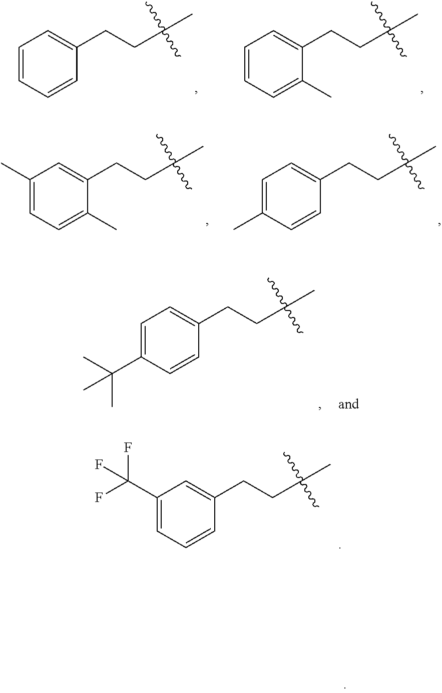

In some embodiments, R¹ is unsubstituted C1-C6 alkyl. In some embodiments, R¹ is

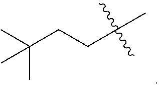 [SMILES: *CCC(C)(C)C]

In some embodiments, R¹ is unsubstituted C1-C4 alkyl. In some embodiments, R¹ methyl, ethyl, propyl, or butyl.

In some embodiments, each R² is independently halogen, cyano, C1-C6 alkyl, C1-C6 alkoxy, or C1-C6 haloalkyl. In some embodiments, one R² is halogen. In some embodiments, one R² is cyano. In some embodiments, one R² is C1-C6 alkyl. In some embodiments, one R² is C1-C6 alkoxy. In some embodiments, one R² is C1-C6 haloalkyl.

In some embodiments, Ring B is phenyl optionally substituted with 1-2 independently selected C1-C6 alkyl. In some embodiments, Ring B is phenyl substituted with 1-2 independently selected C1-C6 alkyl. In some embodiments, Ring B is phenyl substituted with 1-2 independently selected C1-C3 alkyl. In some embodiments, Ring B is phenyl.

In some embodiments, Ring B is 5-6 membered heteroaryl optionally substituted with 1-2 independently selected C1-C6 alkyl. In some embodiments, Ring B is 5-6 membered heteroaryl optionally substituted with 1-2 independently selected C1-C3 alkyl. In some embodiments, Ring B is 5-6 membered heteroaryl substituted with 1-2 independently selected C1-C6 alkyl. In some embodiments, Ring B is unsubstituted 5-6 membered heteroaryl. In some embodiments, Ring B is selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, thiatriazolyl, pyridinyl, pyrimidinyl, pyrazinyl, pyridazinyl, and triazinyl. In some embodiments, Ring B is

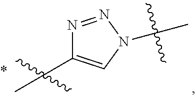 [SMILES: *c1cn(*)nn1]

where the asterisk indicates the point of attachment to Q.

In some embodiments, Ring B is 4-10 membered heterocyclyl optionally substituted with 1-2 independently selected C1-C6 alkyl. In some embodiments, Ring B is 4-10 membered heterocyclyl optionally substituted with 1-2 independently selected C1-C3 alkyl. In some embodiments, Ring B is 5-7 membered heterocyclyl optionally substituted with 1-2 independently selected C1-C6 alkyl. In some embodiments, Ring B is 5-7 membered heterocyclyl optionally substituted with 1-2 independently selected C1-C3 alkyl. In some embodiments, Ring B is 6-7 membered heterocyclyl optionally substituted with 1-2 independently selected C1-C3 alkyl. In some embodiments, Ring B is unsubstituted 6-7 membered heterocyclyl.

In some embodiments, Ring B is selected from the group consisting of

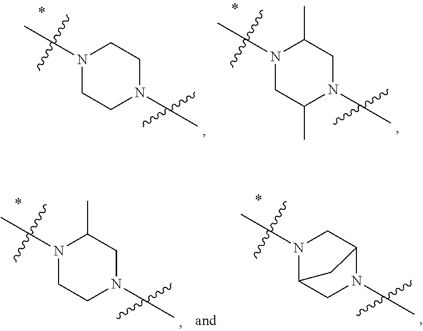

where the asterisk indicates the point of attachment to Q.

In some embodiments, R³ is —C(═O)NR^{A}.

In some embodiments, R³ is —C(═O)OR^{A}.

In some embodiments, R³ is —NH(C═O)R^{A}.

In some embodiments, R³ is —NHC(═O)NR^{A}.

In some embodiments, R³ is C1-C6 alkyl(NHC(═O)NH)R^{A}. In some embodiments, R³ is C1-C3 alkyl(NHC(═O)NH)R^{A}. In some embodiments, R³ is —CH₂CH₂(NHC(═O)NH)R^{A}. In some embodiments, R³ is —CH₂CH(CH₃)(NHC(═O)NH)R^{A}.

In some embodiments, R^{A} is phenyl optionally substituted with 1-2 independently selected R^{A1}. In some embodiments, R^{A} is phenyl substituted with 1-2 independently selected R^{A1}. In some embodiments, R^{A} is phenyl substituted with R^{A1}. In some embodiments, R^{A} is selected from the group consisting of

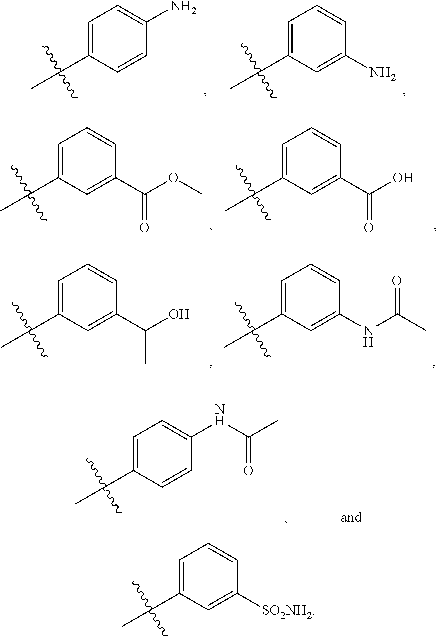

In some embodiments, R^{A} is unsubstituted phenyl.

In some embodiments, R^{A} is 5-6 membered heterocyclyl optionally substituted with 1-2 independently selected R^{A1}. In some embodiments, R^{A} is 5-6 membered heterocyclyl optionally substituted with R^{A1}. In some embodiments, R^{A} is 5-6 membered heterocyclyl optionally substituted with 2 independently selected R^{A1}. In some embodiments, R^{A} is unsubstituted 5-6 membered heterocyclyl. In some embodiments, R^{A} is selected from the group consisting of pyrrolidinyl, tetrahydrofuryl, thiolanyl, pyrazolinyl, oxathiolanyl, isoxazolidinyl, isothiazolidinyl, pyrrolinyl, pyrrolidinonyl, pyrazolidinyl, imidazolinyl, dioxolanyl, sulfolanyl, thiazolidedionyl, succinimidyl, dihydrofuranonyl, pyrazolidinonyl, oxazolidinyl, isoxazolidinonyl, hydantionyl, thiohydantionyl, imidazolidinonyl, oxazolidinonyl, thiazolidinonyl, oxathiolanonyl, dioxolanonyl, dioxazolidinonyl, oxadiazolidinonyl, triazolidinonyl, triazolidinethionyl, oxadiazolidinethionyl, dioxazolidinethionyl, dioxolanethionyl, oxazolidinethionyl, imidazolidinethionyl, isothiazolidinonyl, piperidinyl, tetrahydropyranyl, thianyl, morpholinyl, thiomorpholinyl, dioxanyl, piperazinyl, dithianyl, oxazinyl, tetrahydropyranonyl, piperidinonyl, dioxanonyl, oxazinanonyl, morpholinonyl, thiomorpholinonyl, piperazinonyl, tetrahydropyrimidinonyl, piperidinedionyl, oxazinanedionyl, dihydropyrimidindione, tetrahydropyridazinonyl, triazinanonyl, oxadiazinanonyl, dioxazinanonyl, morpholinedionyl, piperazinedionyl, piperazinetrionyl, and triazinanedionyl.

In some embodiments, R^{A} is 5-6 membered heteroaryl optionally substituted with 1-2 independently selected R^{A1}. In some embodiments, R^{A} is 5-6 membered heteroaryl substituted with 1-2 independently selected R^{A1}. In some embodiments, R^{A} is 5-6 membered heteroaryl substituted with R^{A1}. In some embodiments, R^{A} is 5-6 membered heteroaryl substituted with 2 independently selected R^{A1}. In some embodiments, R^{A} is unsubstituted 5-6 membered heteroaryl. In some embodiments, R^{A} is 5-6 membered heteroaryl selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl, pyridinyl, pyrimidinyl, pyrazinyl, pyridazinyl, and triazinyl. In some embodiments, R^{A} is selected from the group consisting of 3-pyridyl, 3-pyridazinyl, 2-pyrimidinyl, and 2-pyrazinyl.

In some embodiments, each R^{A1} is independently —NR^{B}R^{C}, C-linked ester, —CO₂H, —S(O₂)NH₂, —NHC(═O)C1-C6 alkyl, or C1-C6 alkyl optionally substituted with hydroxyl. In some embodiments, one R^{A1} is —R^{B}R^{C}. In some embodiments, one R^{A1} is —NH₂. In some embodiments, one R^{A1} is —CO₂H. In some embodiments, one R^{A1} is —S(O₂)NH₂. In some embodiments, one R^{A1} is C-linked ester. In some embodiments, one R^{A1} is —CO₂C1-C6 alkyl. In some embodiments, one R^{A1} is —CO₂CH₃.

In some embodiments, one R^{A1} is NHC(═O)C1-C6 alkyl. In some embodiments, one R^{A1} is NHC(═O)C1-C3 alkyl. In some embodiments, one R^{A1} is NHC(═O)CH₃.

In some embodiments, one R^{A1} is C1-C6 alkyl optionally substituted with hydroxyl. In some embodiments, one R^{A1} is C1-C6 alkyl substituted with hydroxyl. In some embodiments, one R^{A1} is C1-C3 alkyl substituted with hydroxyl. In some embodiments, one R^{A1} is

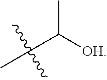 [SMILES: *C(C)O]

In some embodiments, R^{B} and R^{C} is independently hydrogen, C1-C6 alkyl, C1-C6 haloalkyl. In some embodiments, R^{B} and R^{C} are both hydrogen. In some embodiments, R^{B} and R^{C} are both C1-C6 alkyl. In some embodiments, R^{B} and R^{C} are both C1-C6 haloalkyl. In some embodiments, one of R^{B} and R^{C} is hydrogen and the other one of R^{B} and R^{C} is C1-C6 alkyl. In some embodiments, one of R^{B} and R^{C} is hydrogen and the other one of R^{B} and R^{C} is C1-C6 haloalkyl. In some embodiments, one of R^{B} and R^{C} is C1-C6 alkyl and the other one of R^{B} and R^{C} is C1-C6 haloalkyl.

In some embodiments, R^{B} and R^{C}, together with the nitrogen to which they are attached form a 4-6 membered heterocyclyl optionally substituted with 1-2 independently selected halogen, C1-C6 alkyl, C1-C6 haloalkyl, hydroxyl, or amino.

In some embodiments, the compound of Formula (I) is Formula (I-a)

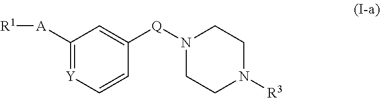

or a pharmaceutically acceptable salt thereof, wherein:
Y is CH or N; and
Q is C(═O), S(═O), S(O₂),

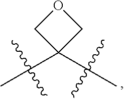 [SMILES: *C1(*)COC1]

or methylene.

In some embodiments, the compound of Formula (I) is Formula (I-b)

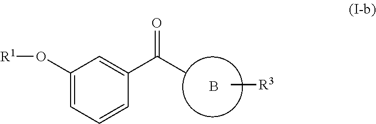

or a pharmaceutically acceptable salt thereof.

In some embodiments, the compound of Formula (I) is Formula (I-c)

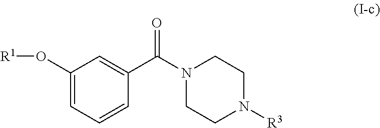

or a pharmaceutically acceptable salt thereof.

In some embodiments, the compound of Formula (I) is selected from Table A, or a pharmaceutically acceptable salt thereof.

TABLE A

| | |
|---|---|
| TM2 | 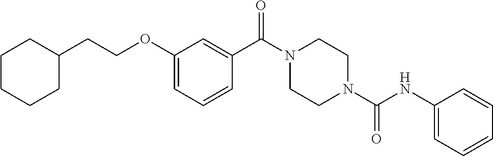 [SMILES: O=C(Nc1ccccc1)N1CCN(C(=O)c2cccc(OCCC3CCCCC3)c2)CC1] |
| TM22 | 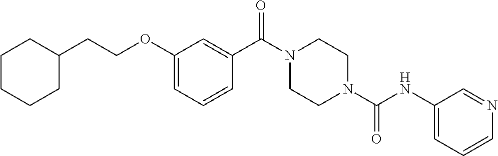 [SMILES: O=C(Nc1cccnc1)N1CCN(C(=O)c2cccc(OCCC3CCCCC3)c2)CC1] |
| TM45 | 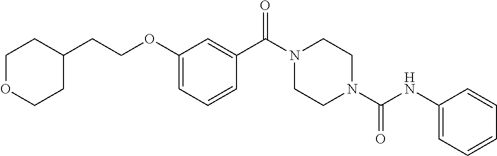 [SMILES: O=C(Nc1ccccc1)N1CCN(C(=O)c2cccc(OCCC3CCOCC3)c2)CC1] |
| TM98 | 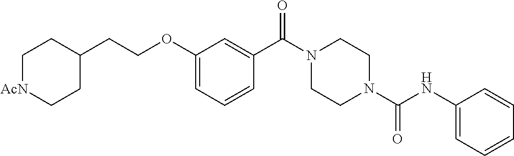 |
| TM112 | 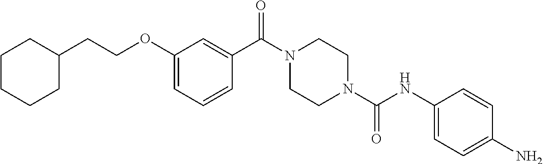 [SMILES: Nc1ccc(NC(=O)N2CCN(C(=O)c3cccc(OCCC4CCCCC4)c3)CC2)cc1] |
| TM70-1 | 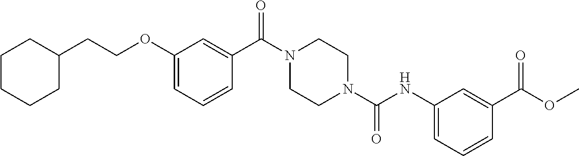 [SMILES: COC(=O)c1cccc(NC(=O)N2CCN(C(=O)c3cccc(OCCC4CCCCC4)c3)CC2)c1] |
| TM70 | 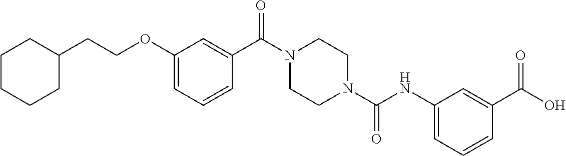 [SMILES: O=C(O)c1cccc(NC(=O)N2CCN(C(=O)c3cccc(OCCC4CCCCC4)c3)CC2)c1] |
| TM91 | 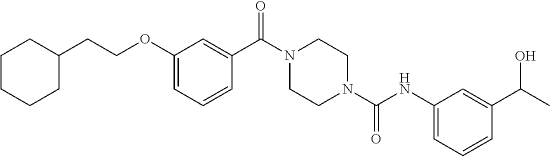 [SMILES: CC(O)c1cccc(NC(=O)N2CCN(C(=O)c3cccc(OCCC4CCCCC4)c3)CC2)c1] |
| TM92 | 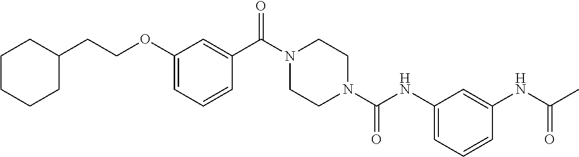 [SMILES: CC(=O)Nc1cccc(NC(=O)N2CCN(C(=O)c3cccc(OCCC4CCCCC4)c3)CC2)c1] |
| TM93 | 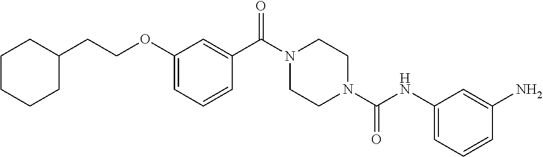 [SMILES: Nc1cccc(NC(=O)N2CCN(C(=O)c3cccc(OCCC4CCCCC4)c3)CC2)c1] |
| TM96 | 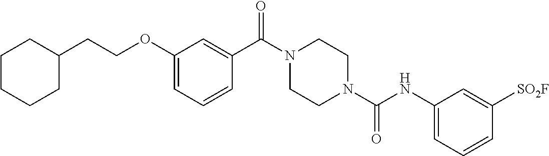 [SMILES: O=C(Nc1cccc(S(=O)(=O)F)c1)N1CCN(C(=O)c2cccc(OCCC3CCCCC3)c2)CC1] |
| TM112-1 | 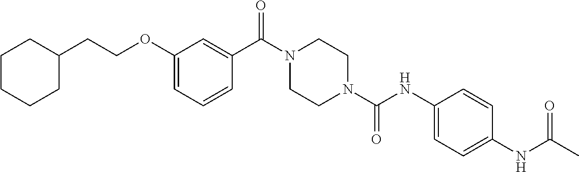 [SMILES: CC(=O)Nc1ccc(NC(=O)N2CCN(C(=O)c3cccc(OCCC4CCCCC4)c3)CC2)cc1] |
| TM88 | 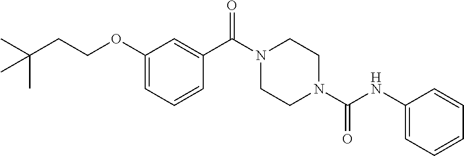 [SMILES: CC(C)(C)CCOc1cccc(C(=O)N2CCN(C(=O)Nc3ccccc3)CC2)c1] |
| TM95 | 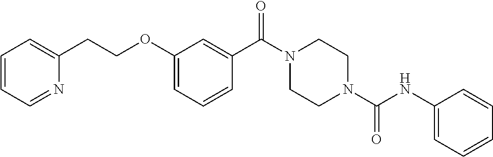 [SMILES: O=C(Nc1ccccc1)N1CCN(C(=O)c2cccc(OCCc3ccccn3)c2)CC1] |
| TM98-1 | 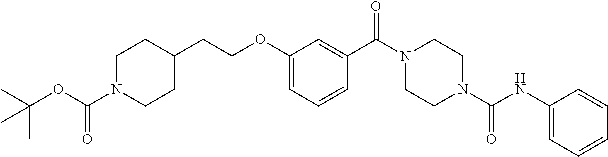 [SMILES: CC(C)(C)OC(=O)N1CCC(CCOc2cccc(C(=O)N3CCN(C(=O)Nc4ccccc4)CC3)c2)CC1] |
| TM98-2 | 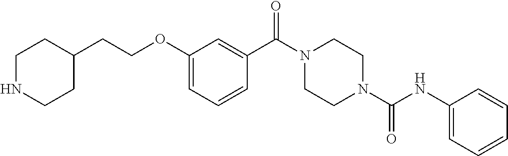 [SMILES: O=C(Nc1ccccc1)N1CCN(C(=O)c2cccc(OCCC3CCNCC3)c2)CC1] |
| TM98 | 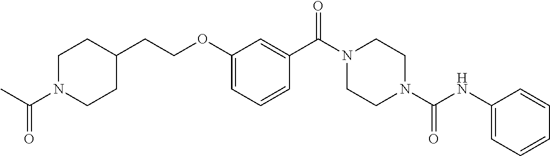 [SMILES: CC(=O)N1CCC(CCOc2cccc(C(=O)N3CCN(C(=O)Nc4ccccc4)CC3)c2)CC1] |
| TM101 | 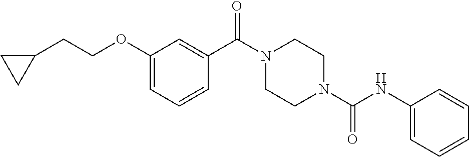 [SMILES: O=C(Nc1ccccc1)N1CCN(C(=O)c2cccc(OCCC3CC3)c2)CC1] |
| TM103 | 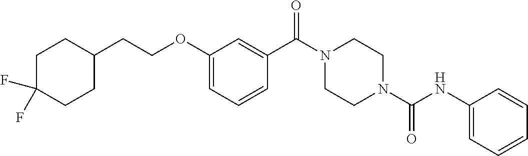 [SMILES: O=C(Nc1ccccc1)N1CCN(C(=O)c2cccc(OCCC3CCC(F)(F)CC3)c2)CC1] |
| TM105 | 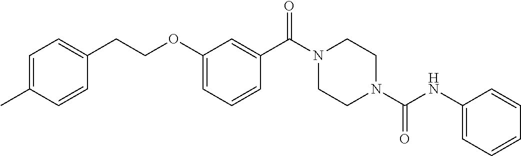 [SMILES: Cc1ccc(CCOc2cccc(C(=O)N3CCN(C(=O)Nc4ccccc4)CC3)c2)cc1] |
| TM106 | 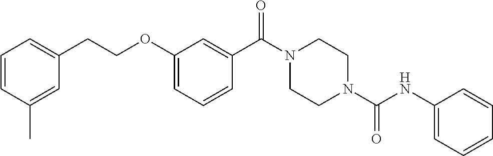 [SMILES: Cc1cccc(CCOc2cccc(C(=O)N3CCN(C(=O)Nc4ccccc4)CC3)c2)c1] |
| TM107 | 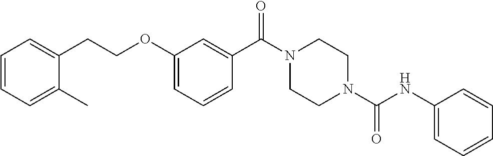 [SMILES: Cc1ccccc1CCOc1cccc(C(=O)N2CCN(C(=O)Nc3ccccc3)CC2)c1] |
| TM108 | 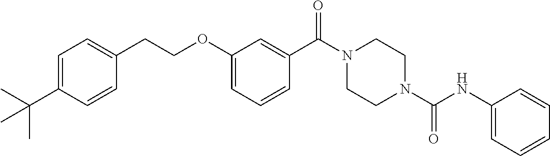 [SMILES: CC(C)(C)c1ccc(CCOc2cccc(C(=O)N3CCN(C(=O)Nc4ccccc4)CC3)c2)cc1] |
| TM111 | 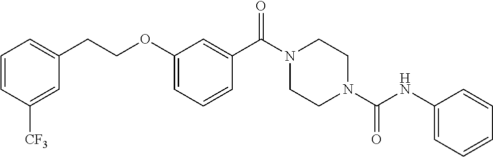 [SMILES: O=C(Nc1ccccc1)N1CCN(C(=O)c2cccc(OCCc3cccc(C(F)(F)F)c3)c2)CC1] |
| TM97 | 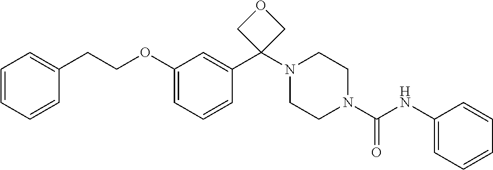 [SMILES: O=C(Nc1ccccc1)N1CCN(C2(c3cccc(OCCc4ccccc4)c3)COC2)CC1] |
| TM99 | 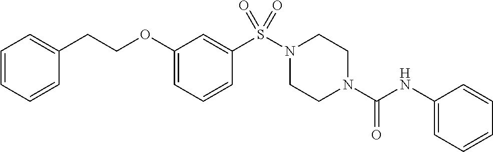 [SMILES: O=C(Nc1ccccc1)N1CCN(S(=O)(=O)c2cccc(OCCc3ccccc3)c2)CC1] |
| TM100 | 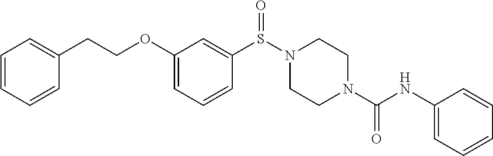 [SMILES: O=C(Nc1ccccc1)N1CCN(S(=O)c2cccc(OCCc3ccccc3)c2)CC1] |
| TM104 | 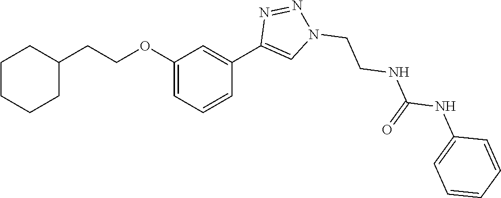 [SMILES: O=C(NCCn1cc(-c2cccc(OCCC3CCCCC3)c2)nn1)Nc1ccccc1] |
| TM109 | 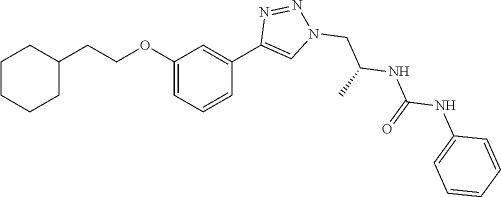 [SMILES: C[C@H](Cn1cc(-c2cccc(OCCC3CCCCC3)c2)nn1)NC(=O)Nc1ccccc1] |
| TM23 | 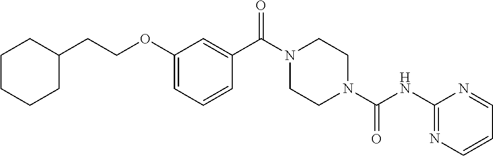 [SMILES: O=C(Nc1ncccn1)N1CCN(C(=O)c2cccc(OCCC3CCCCC3)c2)CC1] |
| TM110 | 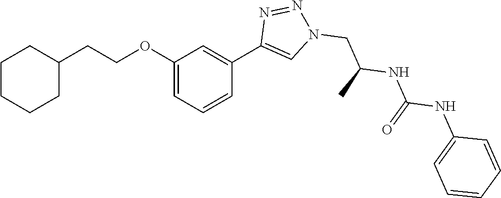 [SMILES: C[C@@H](Cn1cc(-c2cccc(OCCC3CCCCC3)c2)nn1)NC(=O)Nc1ccccc1] |
| MGH-CP-714 | 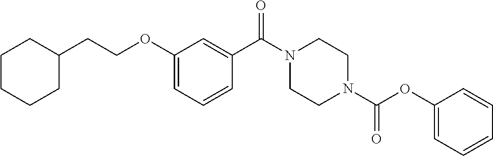 [SMILES: O=C(Oc1ccccc1)N1CCN(C(=O)c2cccc(OCCC3CCCCC3)c2)CC1] |
| MGH-CP-718 | 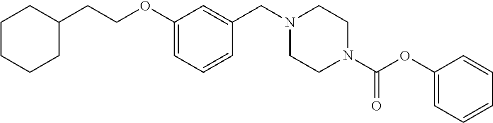 [SMILES: O=C(Oc1ccccc1)N1CCN(Cc2cccc(OCCC3CCCCC3)c2)CC1] |
| MGH-CP-715 | 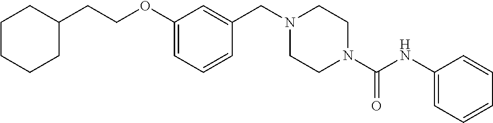 [SMILES: O=C(Nc1ccccc1)N1CCN(Cc2cccc(OCCC3CCCCC3)c2)CC1] |
| MGH-CP-709 | 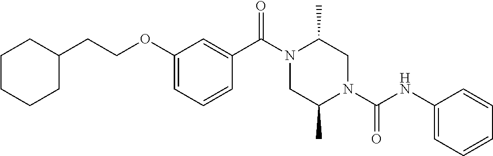 [SMILES: C[C@@H]1CN(C(=O)Nc2ccccc2)[C@@H](C)CN1C(=O)c1cccc(OCCC2CCCCC2)c1] |
| MGH-CP-710 | 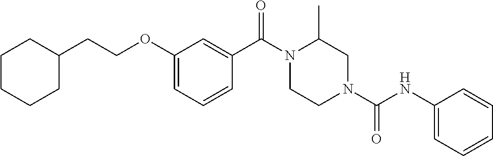 [SMILES: CC1CN(C(=O)Nc2ccccc2)CCN1C(=O)c1cccc(OCCC2CCCCC2)c1] |
| MGH-CP-717 | 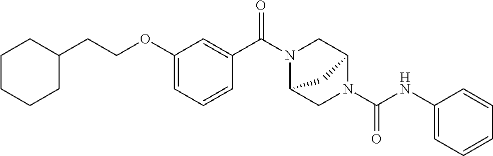 [SMILES: O=C(Nc1ccccc1)N1C[C@@H]2C[C@H]1CN2C(=O)c1cccc(OCCC2CCCCC2)c1] |
| MGH-CP-716 | 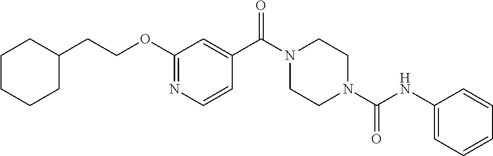 [SMILES: O=C(Nc1ccccc1)N1CCN(C(=O)c2ccnc(OCCC3CCCCC3)c2)CC1] |

### Compositions

Some embodiments provide a composition comprising a compound of Formula (I), or a pharmaceutically acceptable salt thereof and at least one pharmaceutically acceptable excipient. In some embodiments, the compound of Formula (I) in the composition, or a pharmaceutically acceptable salt thereof, is a small molecule inhibitor of TEAD-YAP.

### Methods

Some embodiments provide a method of treating cancer in a subject in need thereof, comprising administering to the subject an effective amount of a compound of Formula (I), or a pharmaceutically acceptable salt thereof, or a pharmaceutical composition comprising a compound of Formula (I), or a pharmaceutically acceptable salt thereof.

In some embodiments, the cancer is medulloblastoma, cutaneous squamous cell carcinoma, lung cancer, pancreatic cancer, esophageal cancer, liver cancer, or colon cancer.

In some embodiments, the cancer is medulloblastoma. In some embodiments, the cancer is cutaneous squamous cell carcinoma. In some embodiments, the cancer is esophageal cancer.

In some embodiments, the cancer is lung cancer, pancreatic cancer, melanoma, liver cancer, or colon cancer.

In some embodiments, the cancer is lung cancer. In some embodiments, the cancer is pancreatic cancer. In some embodiments, the cancer is melanoma. In some embodiments, the cancer is liver cancer. In some embodiments, the cancer is colon cancer. In some embodiments, the compound of Formula (I), or a pharmaceutically acceptable salt thereof, inhibits PDL1 expression and function as immune checkpoint blockade.

### Definitions

The term “n-membered” where n is an integer typically describes the number of ring-forming atoms in a moiety where the number of ring-forming atoms is n. For example, piperidinyl is an example of a 6-membered heterocyclyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted with the indicated groups. The substituents are independently selected, and substitution may be at any chemically accessible position. As used herein, the term “substituted” means that a hydrogen atom is removed and replaced by the indicated substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. The carbon atoms of a “Cₙ₋ₘ alkyl” group can be optionally substituted by one or more oxo (e.g., C(═O)).

As used herein, the term “Cₙ₋ₘ haloalkyl” refers to an alkyl group of Cₙ₋ₘ carbons where one or more hydrogens have been replaced with a halogen. Example haloalkyl groups include trifluormethyl, difluoromethyl, and —CH₂CF₃.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “amino” refers to —NH₂.

As used herein, the term “Cₙ₋ₘ alkylene” refers to a divalent alkyl (e.g., C₁ alkylene is —CH₂—). The alkylene group can be linear, or branched.

As used herein, “halo” or “halogen” refers to F, Cl, Br, or I. In some embodiments, a halo is F, Cl, or Br.

As used herein, the terms “carbonyl” or “oxo”, employed alone or in combination with other terms, refers to a —C(O)— group.

As used herein, the term “acyl” refers to —C(═O)CH₃.

As used herein, “heteroaryl” refers to a monocyclic or polycyclic (e.g., having 2, 3, or 4 fused rings) aromatic heterocycle having at least one heteroatom ring member selected from N, O, S, and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-6 monocyclic heteroaryl having 1, 2, or 3 heteroatom ring members independently selected from N, O, S, and B. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, S, and B. In some embodiments, the heteroaryl group contains 3 to 14, 4 to 14, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridine, pyrimidine, pyrazine, pyridazine, pyrrole, pyrazole, azolyl, oxazole, isoxazole, thiazole, isothiazole, imidazole, furan, thiophene, triazole, tetrazole, thiadiazole, quinoline, isoquinoline, indole, benzothiophene, benzofuran, benzisoxazole, imidazo[1, 2-b]thiazole, purine, triazine, thieno[3,2-b]pyridine, imidazo[1,2-a]pyridine, 1,5-naphthyridine, 1H-pyrazolo[4,3-b]pyridine, and the like.

A five-membered heteroaryl is a heteroaryl group having five ring-forming atoms wherein one or more (e.g., 1, 2, or 3) of the ring-forming atoms are independently selected from N, O, B, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine.

A six-membered heteroaryl ring is a heteroaryl with a ring having six ring-forming atoms wherein one or more (e.g., 1, 2, or 3) ring atoms are independently selected from N, O, S, and B. Exemplary six-membered ring heteroaryls are pyridyl, pyrazinyl, pyrimidinyl, triazinyl and pyridazinyl.

As used herein, “heterocyclyl” refers to monocyclic or polycyclic heterocycles having at least one non-aromatic ring (saturated or partially saturated ring), wherein one or more of the ring-forming carbon atoms of the heterocyclyl is replaced by a heteroatom selected from N, O, S, and B, and wherein the ring-forming carbon atoms and heteroatoms of a heterocyclyl group can be optionally substituted by one or more oxo or sulfide (e.g., C(O), S(O), C(S), or S(O)₂, etc). Heterocyclyl groups include monocyclic and polycyclic (e.g., having 2, 3, or 4 fused rings) systems. Included in heterocyclyl are monocyclic and polycyclic 3-14-, 4-14-, 3-10-, 4-10-, 5-10-4-7-, 5-7-, 5-6-, 5- or 6-membered heterocyclyl groups. Heterocyclyl groups can also include spirocycles and bridged rings (e.g., a 5-14 membered bridged biheterocyclyl ring having one or more ring-forming carbon atoms replaced by a heteroatom independently selected from N, O, S, and B). The heterocyclyl group can be attached through a ring-forming carbon atom or a ring-forming heteroatom. In some embodiments, the heterocyclyl group contains 0 to 3 double bonds, i.e., is partially saturated. In some embodiments, the heterocyclyl group contains 0 to 2 double bonds.

Example heterocyclyl groups include pyrrolidonyl, pyrrolidin-2-one, 1,3-isoxazolidin-2-one, pyranyl, tetrahydropyran, oxetanyl, azetidinyl, morpholinyl, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxabicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl and the like. In some embodiments, the heterocyclyl group is pyrrolidonyl, pyrrolidin-2-one, 1,3-isoxazolidin-2-one, pyranyl, tetrahydropuran, oxetanyl, azetidinyl, morpholinyl, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, or azepanyl.

In some embodiments, the heterocyclyl group contains 3 to 14 ring-forming atoms, 4 to 14 ring-forming atoms, 3 to 7 ring-forming atoms, or 5 to 6 ring-forming atoms. In some embodiments, the heterocyclyl group has 1 to 4 heteroatoms, 1 to 3 heteroatoms, 1 to 2 heteroatoms or 1 heteroatom. In some embodiments, the heterocyclyl is a monocyclic 4-6 membered heterocyclyl having 1 or 2 heteroatoms independently selected from N, O, S, and B and having one or more oxidized ring members. In some embodiments, the heterocyclyl is a monocyclic or bicyclic 4-10 membered heterocyclyl having 1, 2, 3, or 4 heteroatoms independently selected from N, O, S, and B and having one or more oxidized ring members.

As used herein, the term “spiroheterocyclyl” refers to a heterocycly group as defined herein, where the points of attachment are geminal. Non-limiting examples of spiroheterocyclyl include

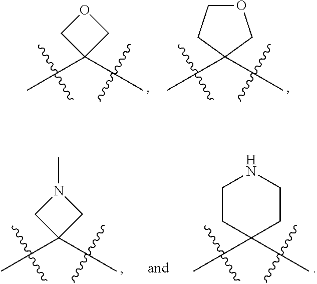

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group. “Oxo” can also refer to an oxygen atom as a ligand to a metal atom, such as an iron atom.

As used herein, the term “C-linked ester” refers to an ester group, i.e., —C(═O)OR linked at the carbonyl. When the point of attachment is to carbon atom, the resulting group is an ester moiety. When the point of attachment is an amine, the resulting moiety is a carbamate.

As used herein, the term “Cₙ₋ₘ cycloalkyl”, refers to a cycloalkyl group made up of from n to m number of carbons. Example cycloalkyl groups include cyclopropyl, cyclobutyl, cyclopentyl, and cyclohexyl.

“Subject” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. In some embodiments, the subject is a human.

The term “about” as used in connection with a numerical value throughout the specification and the claims denotes an interval of accuracy, familiar and acceptable to a person skilled in the art. Such interval of accuracy is, for example, +10%.

An “effective amount” as used herein refers to an amount of an active ingredient or component (e.g., a compound of Formula (I), or a pharmaceutically acceptable salt thereof) that elicits the desired biological or medicinal response in a subject.

Metal cations can include a metal cations with an atomic number of 21-29, 40, 42, or 57-83. For example, metal cations can include stable or unstable isotopes of metals. Metal cations can include mixtures of isotopes or a single isotope. In some embodiments, the metal cation is radioactive. In some embodiments, the metal cation is non-radioactive.

### EXAMPLES

The following examples are illustrative and not intended to be limiting.

### Materials and Methods

### Inhibition of TEAD2 and TEAD4 Auto-Palmitoylation In Vitro

Recombinant 6×His-TEAD protein was treated with Compounds under indicated concentrations in 50 mM MES buffer (PH 6.4) for 30 mins. After incubation with 1 μM of alkyne palmitoyl-CoA (15968, Cayman) for 1 h, 50 μL of sample mixture was treated with 5 μL of freshly prepared “click” mixture containing 100 uM TBTA (678937, Sigma-Aldrich), 1 mM TCEP (C4706, Sigma-Aldrich), 1 mM CuSO₄ (496130, Sigma-Aldrich), 100 uM Biotin-Azide (1167-5, Click Chemistry Tools) and incubated for another 1 h. The samples were then added 11 μL of 6×SDS loading buffer (BP-11 IR, Boston BioProducts) and denatured at 95° C. for 5 mins. SDS-PAGE was used to analyze the samples. Palmitoylation signal was detected by streptavidin-HRP antibody (1:3000, S911, Invitrogen). The total protein level was detected by primary anti-His-tag antibody (1:10000, MA1-21315, Invitrogen) and secondary anti-mouse antibodies (1:5000, 7076S, Cell Signaling). The band intensities were quantified with ImageJ. The inhibition of auto-palmitoylation by compounds were normalized to DMSO. The IC₅₀ curves were plotted with GraphPad prism6.

### Cell Culture

Human H226, MSTO-211H, H2052, H28, HCT116, DLD1 cells were obtained from ATCC (Manassas, VA). HEK293A, HCT116, DLD1 cells were cultured in Dulbecco's modified Eagles media (DMEM) (Life Technologies) supplemented with 10% (v/v) fetal bovine serum (FBS) (Thermo/Hyclone, Waltham, MA), 100 units/mL penicillin and 100 μg/mL streptomycin (Life technologies) at 37° C. with 5% C02. H226, MSTO-211H, H2052, H28 cells were cultured in RPMI 1640 medium (Life technologies) supplemented with 10% (v/v) fetal bovine serum (FBS) (Thermo/Hyclone, Waltham, MA), 100 units/mL penicillin, 100 μg/mL streptomycin (Life technologies), 2.5 g/L glucose and 1 mM sodium pyruvate at 37° C. with 5% C02.

### Transfection

HEK293A cells were seeded in 6 cm dishes overnight and transfected with plasmids using PEI reagent (1 μg/μL). Briefly, PRK5-Myc-TEAD1 (33109, Addgene) and PEI were diluted in serum-free DMEM medium in two tubes (DNA: PEI ratio=1:2). After standing still for 5 mins, mix them well and stay for another 20 mins. The mixture was then added to dishes directly.

### Inhibition of TEAD Palmitoylation in HEK293A Cells

HEK293A cells with or without TEAD overexpression were pretreated with DMSO or TM2 in medium with 10% dialyzed fetal bovine serum (DFBS) for 8 h and labeled by Alkynyl Palmitic acid (1165, Click Chemistry Tools) for another 16 h. The cells were then washed and harvested by cold DPBS (14190250, Life Technologies). The cell pellets were isolated by centrifugation (500×g, 10 min) and lysed by TEA lysis buffer (50 mM TEA-HCl, pH 7.4, 150 mM NaCl, 1% Triton X-100, 0.2% SDS, 1×Protease inhibitor-EDTA free cocktail (05892791001, Roche), phosphatase inhibitor cocktail (P0044, Sigma-Aldrich)) on ice for 30 mins. The protein concentration was determined using Bio-Rad assay and adjusted to 1 mg/mL. 100 μL of protein sample mixture was treated with 10 μL of freshly prepared “click” mixture containing 1 mM TBTA, 10 mM TCEP, 10 mM CuSO₄, 1 mM TBTA Biotin-Azide and incubated for 1 h at room temperature. The proteins were precipitated by chloroform/methanol/H20 mixture and redissolved with 2% SDS in 0.1% PBST. The solution was diluted with 0.1% PBST and incubated with prewashed streptavidin agarose beads (69203-3, E M D MILLIPORE). After rotation at room temperature for 2 h, the beads were then pelleted by centrifugation (500×g, 3 min) and washed with 0.2% SDS in PBS (3×1 mL). The bound proteins were eluted with a buffer containing 10 mM EDTA pH 8.2 and 95% formamide and analyzed with SDS-PAGE. Anti-Myc (1:1000, 2278S, Cell Signaling) or anti-pan-TEAD (1:1000, 13295, Cell Signaling) antibody were used to detect Myc-TEAD1 or pan-TEAD, respectively. Secondary antibody was anti-rabbit (1:5000, 7074S, Cell Signaling).

### Protein Purification, Crystallization, and Structure Determination

The recombinant human TEAD2 (residues 217-447, TEAD2 217-447) protein was purified and crystallized as described previously (Li et al., 2020b). Single crystals were soaked overnight at 20° C. with 5 mM TM2, 5% DMSO in reservoir solution supplemented with 25% glycerol and flashed-cooled in liquid nitrogen. Diffraction data was collected at beamline 19-ID (SBC-XSD) at the Advanced Pho-ton Source (Argonne National Laboratory) and processed with HKL3000 program (Otwinowski and Minor, 1997). Best crystals diffracted 2.40 Å and exhibited the symmetry of space group C2 with cell dimensions of a=124.1 Å, b=62.3 Å, c=79.9 Å and β=117.7°. Using TEAD2 structure (PDB ID: 3L15) as searching model, initial density map and model were generated by molecular replacement with Phaser in PHENIX (Adams et al., 2010). There are two TEAD2 molecules in the asymmetric unit. One TM2 molecule was built in the cavity of each TEAD2 molecule, and the remaining residues were manually built in COOT39 and refined in PHENIX. The final model (Rwork=0.184, Rfree=0.235) contains 400 residues, 30 water molecules and two TM2 molecules. Statistics for data collection and structure refinement are summarized in Table 1. Structural analysis and generation of graphics were carried out in PyMOL.

### Co-Immunoprecipitation (Co-IP) Assay

H226 cells were treated with DMSO or TM2 for 24 h. The cells were then washed and harvested by cold DPBS. The cell pellets were isolated by centrifugation (500×g, 10 min) and lysed by lysis buffer (50 mM Tris-HCl pH 7.5, 10% Glycerol, 1% NP-40, 300 mM NaCl, 150 mM KCl, 5 mM EDTA, phosphatase inhibitor cocktail, complete EDTA-free protease inhibitors cocktail) on ice. After dilution with 50 mM Tris-HCl pH 7.5, 10% Glycerol, 1% NP-40, 5 mM EDTA, the protein samples were incubated with mouse anti-YAP antibody (sc-101199, Santa Cruz) overnight at 4° C. and immunoprecipitated with prewashed protein A/G beads (P5030-1, UBPBio) for another 4 h at 4° C. The bound proteins were washed with 0.1% PBST for three times and eluted with 1×SDS loading buffer and analyzed with SDS-PAGE. Anti-TEAD1 (1:1000, 12292S, Cell Signaling), anti-pan-TEAD (1:1000, 13295, Cell Signaling) or anti-YAP (1:1000, 140745, Cell Signaling) antibody were used to detect TEAD1, pan-TEAD or YAP, respectively. Secondary antibody was anti-rabbit (1:5000, 7074S, Cell Signaling).

### Quantitative RT-PCR

H226 cells were treated with DMSO or TM2 for 24 h and used to extract RNA using the RNeasy mini kit (74104, Qiagen). The high-capacity cDNA reverse transcription kit (4368814, Life Technologies) was employed to obtain cDNA. Target genes expression (Cyr61, CTGF and ANKRD1) was measured with PowerUp SYB Green Master Mix kit (A25777, Life Technologies). β-actin was used as reference gene. The primers are shown below:

hCyr61
Forward
GGAAAAGGCAGCTCACTGAAGC
Reverse
GGAGATACCAGTTCCACAGGTC
hCTGF
Forward
CTTGCGAAGCTGACCTGGAAGA
Reverse
CCGTCGGTACATACTCCACAGA
hANKRD1
Forward
CGACTCCTGATTATGTATGGCGC
Reverse
GCTTTGGTTCCATTCTGCCAGTG
hß-actin
Forward
CACCATTGGCAATGAGCGGTTC
Reverse
AGGTCTTTGCGGATGTCCACGT

### RNA-Seq Analysis

The NCI-H226 cells were treated with TM2 at 1 μM for 24 hours. Total RNA was isolated with RNeasy Mini Kit (74104, Qiagen). The integrity of isolated RNA was analyzed using Bioanalyzer (Agilent Technologies). and the RNA-seq libraries were made by Novogene. All libraries have at least 50 million reads sequenced (150 bp paired-end). The heatmaps were generated using different expressed genes from TM2 treatment in NCI-H226 cells with Motpheus (https://software.broadinstitute.org/morpheus/). Principle component analysis (PCA) was determined by PCA function in M3C package in R. Gene Set Enrichment Analysis (GSEA) was performed using GSEA software from Broad Institute (http://software.broadinstitute.org/gsea/index.jsp). The YAP_TAZ-TEAD Direct Target Genes set were generated with the published YAP/TAZ-TEAD target genes (Zanconato et al., 2015).

### Cell Proliferation Assay

H226, MSTO-211H, H2052, H28, HCT116 and DLD1 cells were seeded at a concentration of 500-2000 cells/well in 100 uL of culture medium in 96 well plates overnight and treated with compounds with 3-fold dilutions of concentrations from 10 μM for 5-7 days. After removal of medium, to each well was added 60 μL of MTT reagent (3-(4,5-dimethylthiazol-2-yl)-2,5-diphenyltetrazolium bromide) followed by incubation under 37° C. for 4 h. The absorbance was measured by PerkinElmer EnVision plate reader.

### Drug Combination

The drug combination experiments were preformed using a drug combination matrix across 5 doses of TM2 (5 μM, 3-fold dilution) and 9 doses of Trametinib (10 μM, 3-fold dilution) in different tumor cell lines. Cell viability was determined at day 5 after the drugs administration by MTT. Drug synergy score was calculated followed Bliss rule. Synergy Score and Plot was generated by “Synergyfinder” package in R language.

### Organoids Viability

Mouse hepatic progenitor organoids (70932, STEMCELL Tech) were seeded in 96 well plate using 20 ul Matrigel (Corning, #354230) and cultured in HepatiCult™ Organoid Growth Medium (06031, STEMCELL Tech) with or without TM2. Medium was replaced after every 48 h with fresh compound. Organoid viability was measured by PrestoBlue™ HS Cell Viability Reagent (ThermoFisher, #P50200) following the manufacturer's protocol.

### Immunofluorescence Staining

Organoids were plated in 8 well chamber slide and fixed in 4% paraformaldehyde at 4° C. for 1h. After permeabilization in 0.5% PBST, organoids were blocked with 2% BSA for 2 h and incubated with primary antibody overnight at 40 C. Imaging was performed on Nikon A1RHD25 confocal microscope.

### Statistics

Data was analyzed by GraphPad prism6 and shown as mean±SEM. All the biochemical experiments are repeated for at least 3 times and shown by representative images. Two-tailed t-test was used for P value calculation.

### Synthesis of TEAD Inhibitors

All commercially available reagents were used without further purification. All solvents such as ethyl acetate, DMSO and Dichloromethane (DCM), were ordered from Fisher Scientific and Sigma-Aldrich and used as received. Unless otherwise stated, all reactions are conducted under air. Analytical thin-layer chromatography (TLC) plates from Sigma were used to monitor reactions. Flash column chromatography was employed for purification and performed on silica gel (230-400 mesh). 1H NMR were recorded at 500 MHZ on JEOL spectrometer. 13C NMR were recorded at 125 MHZ on JEOL spectrometer. The chemical shifts were determined with residual solvent as internal standard and reported in parts per million (ppm).

### Example 1. Synthesis of TM2

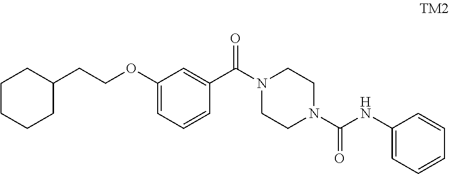 [SMILES: O=C(Nc1ccccc1)N1CCN(C(=O)c2cccc(OCCC3CCCCC3)c2)CC1]

### Step 1: Methyl 3-(2-cyclohexylethoxy)benzoate (S3)

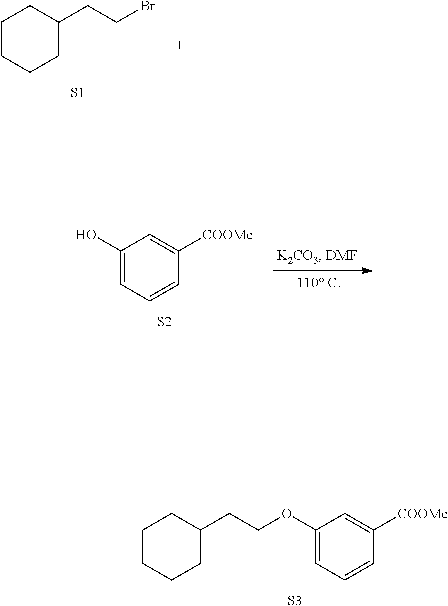

To a solution of methyl 3-hydroxybenzoate S2 (500 mg, 3.29 mmol) in DMF (7 mL) was added (2-bromoethyl)cyclohexane S1 (628.8 mg, 3.29 mmol) and K2CO₃ (628.1 mg, 4.94 mmol). The mixture was then stirred at 110° C. for 4 h. After cooling to temperature, the reaction mixture was diluted with water and extracted with Ethyl acetate. The combined organic layer was washed with brine, dried over Na₂SO₄ and concentrated in vacuo. The crude residue was purified through silica gel chromatography to give S3 as colorless oil (780 mg, 90%). ¹H NMR (500 MHz, Chloroform-d) δ 7.61 (d, J=7.6 Hz, 1H), 7.55 (t, J=2.1 Hz, 1H), 7.33 (t, J=7.9 Hz, 1H), 7.09 (dd, J=8.2, 2.6 Hz, 1H), 4.03 (t, J=6.7 Hz, 2H), 3.91 (s, 3H), 1.83-1.63 (m, 7H), 1.51 (ttt, J=10.5, 6.8, 3.5 Hz, 1H), 1.33-1.11 (m, 3H), 0.98 (qd, J=11.9, 3.3 Hz, 2H).

### Step 2: 3-(2-Cyclohexylethoxy)benzoic acid (S4)

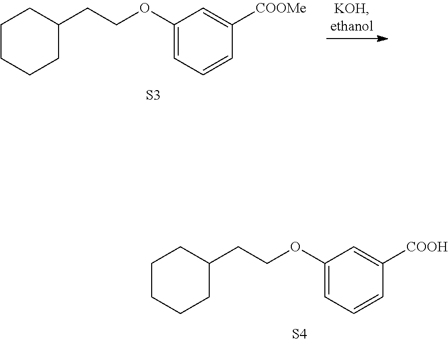 [SMILES: COC(=O)c1cccc(OCCC2CCCCC2)c1 ; "S3" ; O=C(O)c1cccc(OCCC2CCCCC2)c1 ; "S4"]

To a solution of S3 (780 mg, 2.97 mmol) in ethanol (10 mL) was added saturated aqueous KOH (417 μL). The mixture was then stirred at room temperature overnight. After completion, the reaction was quenched with 1 N HCl on ice until pH was adjusted to 1. The mixture was then diluted with water and extracted with ethyl acetate. The combined organic layer was washed with brine, dried over anhydrous Na₂SO₄ and concentrated in vacuo to give S4 (650 mg, 88%) which were used directly without further purification.

### Step 3: tert-Butyl 4-(3-(2-cyclohexylethoxy)benzoyl)piperazine-1-carboxylate (S5)

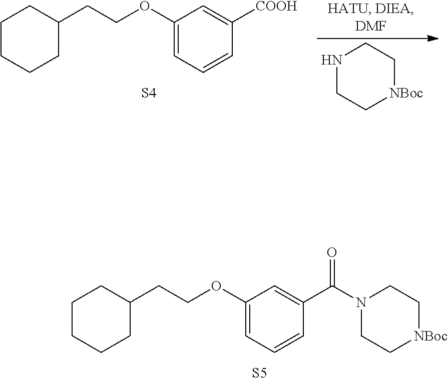 [SMILES: O=C(O)c1cccc(OCCC2CCCCC2)c1 ; "S4" ; CC(C)(C)OC(=O)N1CCNCC1 ; CC(C)(C)OC(=O)N1CCN(C(=O)c2cccc(OCCC3CCCCC3)c2)CC1 ; "S5"]

To a solution of S4 (600 mg, 2.42 mmol) in DMF (20 mL) was added HATU (1.38 g, 3.63 mmol) and DIEA (862 μL, 4.84 mmol). After stirring for 5 mins, a solution of tert-butyl piperazine-1-carboxylate (450.6 mg, 2.42 mmol) was added and the reaction mixture was continuously stirred at room temperature overnight. After completion, the reaction was quenched with water and extracted with ethyl acetate. The combined organic layer was washed with 1 N HCl, saturated NaHCO₃, brine, dried over anhydrous Na₂SO₄ and concentrated in vacuo. The crude residue was purified through silica gel chromatography to give S5 as a white solid (950 mg, 94%). ¹H NMR (500 MHz, Chloroform-d) δ 7.30 (t, J=8.0 Hz, 1H), 6.96-6.89 (m, 3H), 3.99 (t, J=6.7 Hz, 2H), 3.82-3.31 (m, 8H), 1.79-1.62 (m, 7H), 1.54-1.39 (m, 1H) 1.47 (s, 9H), 1.32-1.10 (m, 3H), 0.96 (qd, J=11.9, 3.0 Hz, 2H).

### Step 4: (3-(2-Cyclohexylethoxy)phenyl)(piperazin-1-yl)methanone (S6)

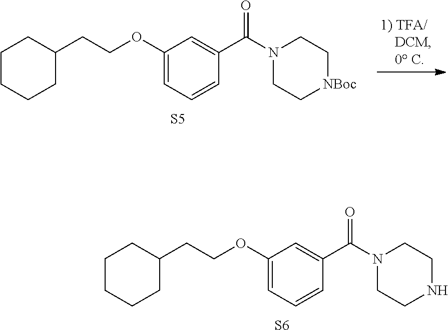 [SMILES: CC(C)(C)OC(=O)N1CCN(C(=O)c2cccc(OCCC3CCCCC3)c2)CC1 ; "S5" ; O=C(c1cccc(OCCC2CCCCC2)c1)N1CCNCC1 ; "S6"]

To a solution of S5 (890 mg, 2.13 mmol) in DCM (4 mL) was added trifluoroacetic acid (4 mL) dropwise on ice. The mixture was continuously stirred on ice for 30 mins. After completion, the reaction was quenched with saturated NaHCO₃ dropwise on ice. The mixture was then diluted with water and extracted with ethyl acetate. The combined organic layer was washed with brine, dried over anhydrous Na₂SO₄ and concentrated in vacuo to give S6 which were used directly without further purification.

### Step 5: 4-(3-(2-cyclohexylethoxy)benzoyl)-N-phenylpiperazine-1-carboxamide (TM2)

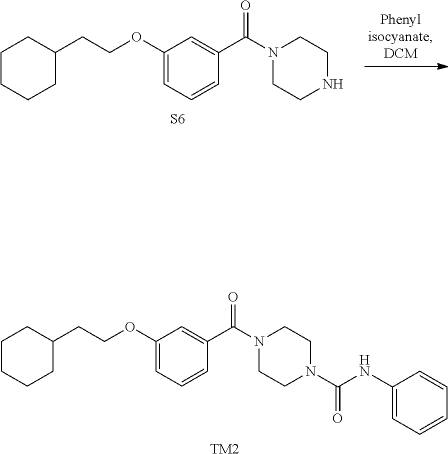

To a solution of S6 (100 mg, 0.403 mmol) in DCM (4 mL) was added phenyl isocyanate (63.1 μL, 0.484 mmol). The reaction mixture was stirred at room temperature for 2 h. The reaction was quenched with water and extracted with DCM. The combined organic layer was washed with brine, dried over anhydrous Na₂SO₄ and concentrated in vacuo. The crude residue was purified through silica gel chromatography to give TM2 as a white solid (160 mg, 91%). ¹H NMR (500 MHz, Chloroform-d) δ 7.36-7.24 (m, 5H), 7.04 (t, J=7.3 Hz, 1H), 6.98-6.89 (m, 3H), 6.77 (brs, 1H), 3.99 (t, J=6.7 Hz, 2H), 3.93-3.35 (m, 8H), 1.78-1.62 (m, 7H), 1.54-1.44 (m, 1H), 1.30-1.12 (m, 3H), 0.97 (qd, J=12.1, 2.9 Hz, 2H). ¹³C NMR (125 MHz, Chloroform-d) δ 170.62, 159.45, 155.21, 138.85, 136.47, 129.85, 129.02, 123.55, 120.41, 118.87, 116.46, 113.17, 66.28, 47.46 (brs), 44.22, 42.01 (brs), 36.64, 34.61, 33.39, 26.60, 26.33.

### Example 2. Synthesis of TM22

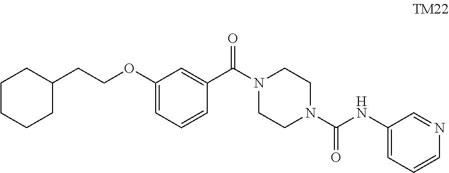 [SMILES: O=C(Nc1cccnc1)N1CCN(C(=O)c2cccc(OCCC3CCCCC3)c2)CC1]

### Step 1: Phenyl pyridin-3-ylcarbamate (S8)

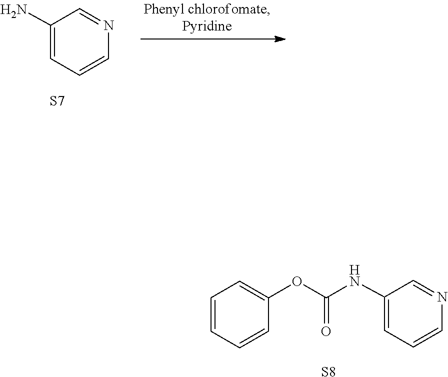

To a solution of Pyridin-3-amine S7 (188.2 mg, 2 mmol) in pyridine (5 mL) was added phenyl chloroformate (274 μL, 2.2 mmol). The reaction mixture was stirred at room temperature overnight. The mixture was quenched by the addition of ethyl acetate and 10% critic acid. The organic layer was washed with saturated NaHCO₃, brine, dried over Na₂SO₄. The organic solvents were removed in vacuo to give carbamate S8 which was used directly for the next step.

### Step 2: 4-(3-(2-cyclohexylethoxy)benzoyl)-N-(pyridin-3-yl)piperazine-1-carboxamide (TM22)

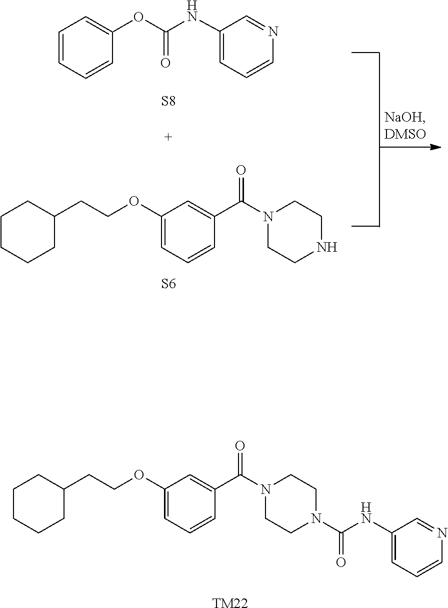

To a solution of S6 (30 mg, 0.095 mmol) in DMSO (1 mL) was added carbamate (40.7 mg, 0.19 mmol) and NaOH (114 μL, 0.114 mmol, 10 N). The reaction mixture was stirred at room temperature for 2 h. The reaction was quenched with water and extracted with ethyl acetate. The combined organic layer was washed with brine, dried over anhydrous Na₂SO₄ and concentrated in vacuo. The crude residue was purified through silica gel chromatography to give TM22 as a white solid (36.1 mg, 87%). ¹H NMR (500 MHz, Chloroform-d) δ 8.46 (d, J=2.6 Hz, 1H), 8.26 (dd, J=4.8, 1.4 Hz, 1H), 7.96 (dt, J=8.4, 2.1 Hz, 1H), 7.34-7.20 (m, 3H), 6.99-6.87 (m, 3H), 3.99 (t, J=6.7 Hz, 2H), 3.88-3.37 (m, 8H), 1.77-1.63 (m, 7H), 1.55-1.45 (m, 1H), 1.29-1.13 (m, 3H), 0.96 (qd, J=12.0, 2.9 Hz, 2H). ¹³C NMR (125 MHz, Chloroform-d) δ 170.70, 159.50, 155.05, 144.25, 141.49, 136.36, 136.25, 129.93, 127.78, 123.78, 118.82, 116.49, 113.21, 66.32, 47.43 (brs), 44.24, 42.00 (brs), 36.65, 34.64, 33.41, 26.62, 26.35.

### Example 3. TM45

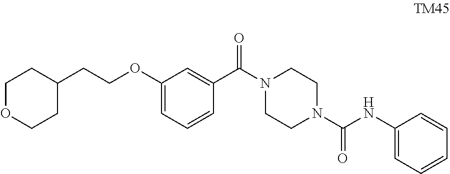 [SMILES: O=C(Nc1ccccc1)N1CCN(C(=O)c2cccc(OCCC3CCOCC3)c2)CC1]

### Step 1: Methyl 3-(2-(tetrahydro-2H-pyran-4-yl)ethoxy)benzoate (S10)

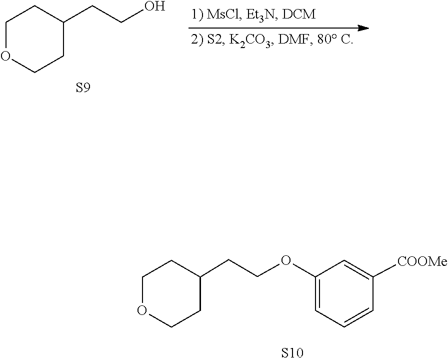

To a solution of S9 (400 mg, 3.07 mmol) in anhydrous DCM (20 mL) was added Et₃N (642 μL, 4.61 mmol), MsCl (285 μL, 3.68 mmol) at 0° C. The solution was stirred at room temperature. After completion, the reaction mixture was diluted with water, extracted with DCM, washed with saturated aqueous NaHCO₃. The combined organic layer was dried over anhydrous Na₂SO₄ and concentrated in vacuo to give the methanesulfonate. The methanesulfonate was then dissolved in DMF (10 mL) followed by addition of S2 (513.8 mg, 3.38 mmol) and K2CO₃ (848.6 mg, 6.14 mmol). The resulting suspension was further stirred at 80° C. for 4 h. The reaction mixture was extracted with ethyl acetate, then washed with water, brine. The organic phase was dried over anhydrous Na₂SO₄ and concentrated in vacuo. The crude residue was purified through silica gel chromatography to give S10 as colorless oil (680 mg, 84%). ¹H NMR (500 MHz, Chloroform-d) δ 7.62 (dd, J=7.5, 1.3 Hz, 1H), 7.54 (t, J=2.1 Hz, 1H), 7.33 (t, J=7.9 Hz, 1H), 7.08 (dd, J=8.4, 2.6 Hz, 1H), 4.05 (t, J=6.2 Hz, 2H), 3.97 (ddd, J=11.4, 4.5, 1.7 Hz, 2H), 3.91 (s, 3H), 3.41 (td, J=11.8, 2.1 Hz, 2H), 1.85-1.73 (m, 3H), 1.67 (dq, J=13.3, 2.0 Hz, 2H), 1.37 (qd, J=11.9, 4.4 Hz, 2H).

### Step 2: 3-(2-(Tetrahydro-2H-pyran-4-yl)ethoxy)benzoic acid (S12)

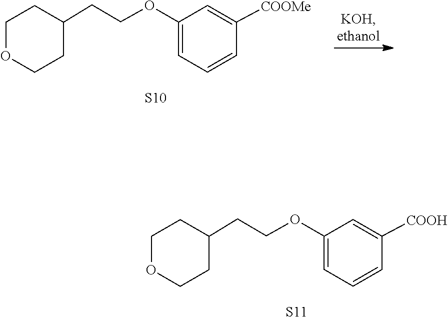

S11 was prepared as described for S4 (670 mg, 2.53 mmol) from S10 and was used directly without further purification.

### Step 3: tert-Butyl 4-(phenylcarbamoyl)piperazine-1-carboxylate (S12)

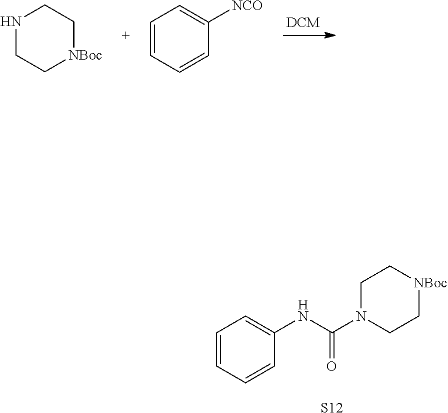

S12 was prepared as described for TM2 from tert-butyl piperazine-1-carboxylate (1 g, 5.37 mmol) and phenyl isocyanate (767.5 mg, 6.44 mmol) as a white solid (quantitative). ¹H NMR (500 MHz, Chloroform-d) δ 7.35 (d, J=7.6 Hz, 2H), 7.29 (td, J=8.5, 8.0, 2.3 Hz, 2H), 7.08-7.02 (m, 1H), 6.37 (s, 1H), 3.49 (s, 8H), 1.49 (s, 9H).

### Step 4: N-phenylpiperazine-1-carboxamide (S13)

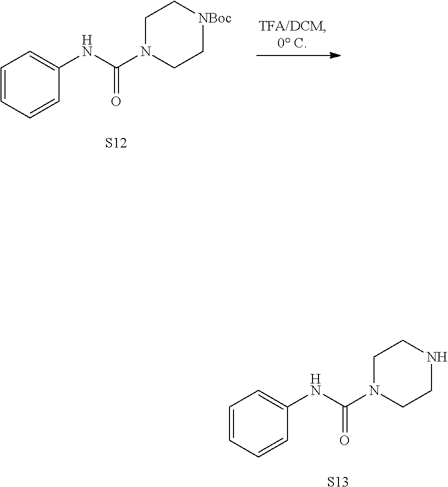 [SMILES: CC(C)(C)OC(=O)N1CCN(C(=O)Nc2ccccc2)CC1 ; "TFA/DCM,0° C." ; "S12" ; O=C(Nc1ccccc1)N1CCNCC1 ; "S13"]

S13 was prepared as described for S6 (800 mg, 2.62 mmol) from S12 and was used directly without further purification.

### Step 5: N-phenyl-4-(3-(2-(tetrahydro-2H-pyran-4-yl)ethoxy)benzoyl)piperazine-1-carboxamide (TM45)

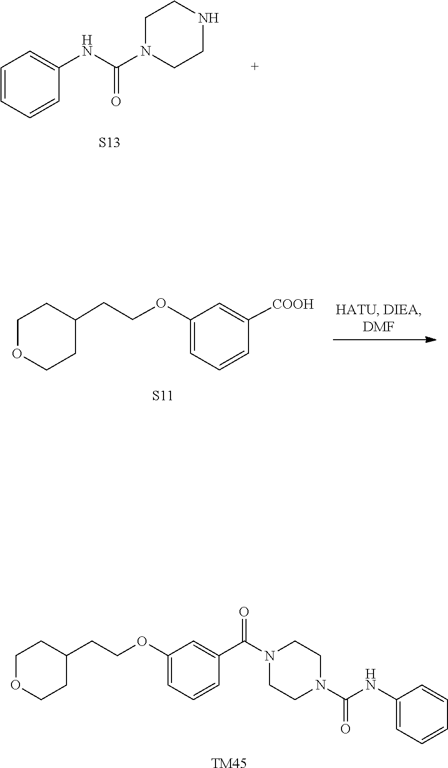

TM45 was prepared as described for S5 from S11 (40 mg, 0.16 mmol) and S13 (39.4 mg, 0.192 mmol) as a white solid (44 mg, 63%). ¹H NMR (500 MHz, Chloroform-d) δ 7.35-7.29 (m, 3H), 7.29-7.24 (m, 2H), 7.07-7.01 (m, 1H), 6.98-6.89 (m, 3H), 6.73 (s, 1H), 4.01 (t, J=6.1 Hz, 2H), 3.96 (dd, J=11.1, 3.6, 2H), 3.86-3.43 (m, 8H), 3.39 (td, J=11.8, 2.0 Hz, 2H), 1.81-1.71 (m, 3H), 1.68-1.61 (m, 2H), 1.40-1.30 (m, 2H). ¹³C NMR (125 MHz, Chloroform-d) δ 170.52, 159.30, 155.19, 138.85, 136.56, 129.88, 129.01, 123.55, 120.37, 119.03, 116.40, 113.17, 68.06, 65.48, 47.43 (brs), 44.21, 42.01 (brs), 36.15, 33.07, 32.01.

### Example 4: Synthesis of TM98

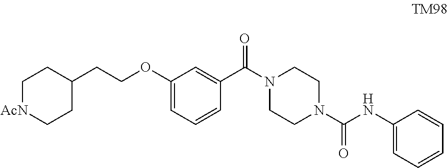

### Step 1: tert-Butyl 4-(2-(3-(methoxycarbonyl)phenoxy)ethyl)piperidine-1-carboxylate (S15)

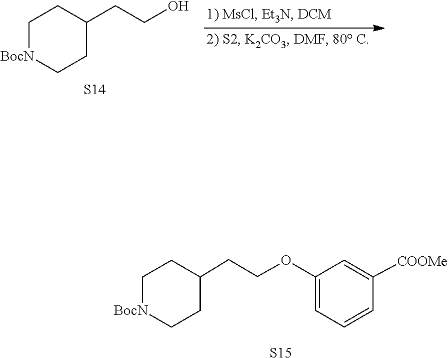 [SMILES: CC(C)(C)OC(=O)N1CCC(CCO)CC1 ; "S14" ; COC(=O)c1cccc(OCCC2CCN(C(=O)OC(C)(C)C)CC2)c1 ; "S15"]

S15 was prepared as described for S10 from S14 (480 mg, 2.09 mmol) and S12 (318 mg, 2.09 mmol) as a white solid (530 mg, 70%). ¹H NMR (500 MHz, Chloroform-d) δ 7.62 (dd, J=7.7, 1.3 Hz, 1H), 7.54 (dd, J=2.7, 1.3 Hz, 1H), 7.33 (t, J=7.9 Hz, 1H), 7.08 (ddd, J=8.3, 2.6, 1.2 Hz, 1H), 4.19-4.05 (m, 2H), 4.05 (t, J=6.1 Hz, 2H), 3.91 (s, 3H), 2.80-2.64 (s, 2H), 1.80-1.65 (m, 5H), 1.46 (s, 9H), 1.23-1.11 (m, 2H).

### Step 2: 3-(2-(1-(tert-Butoxycarbonyl)piperidin-4-yl)ethoxy)benzoic acid (S16)

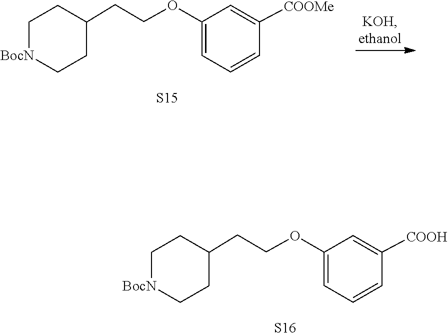 [SMILES: COC(=O)c1cccc(OCCC2CCN(C(=O)OC(C)(C)C)CC2)c1 ; "S15" ; CC(C)(C)OC(=O)N1CCC(CCOc2cccc(C(=O)O)c2)CC1 ; "S16"]

S16 was prepared as described for S4 from S15 (380 mg, 1.05 mmol) and was used directly without further purification.

### Step 3: tert-Butyl 4-(2-(3-(4-(phenylcarbamoyl)piperazine-1-carbonyl)phenoxy)ethyl)piperidine-1-carboxylate (S17)

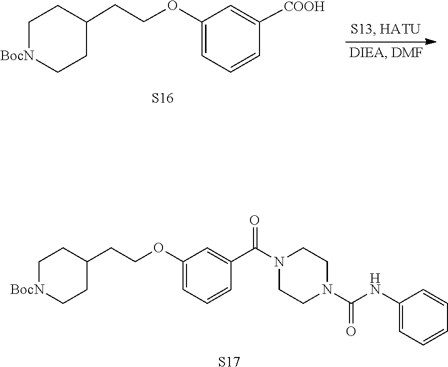 [SMILES: CC(C)(C)OC(=O)N1CCC(CCOc2cccc(C(=O)O)c2)CC1 ; "S16" ; CC(C)(C)OC(=O)N1CCC(CCOc2cccc(C(=O)N3CCN(C(=O)Nc4ccccc4)CC3)c2)CC1 ; "S17"]

S17 was prepared as described for S5 from S16 (200 mg, 0.572 mmol) and S13 (140.9 mg, 0.686 mmol) as a white solid (270 mg, 88%). ¹H NMR (500 MHz, Chloroform-d) δ 7.33 (t, J=7.9 Hz, 3H), 7.30-7.24 (m, 2H), 7.03 (tt, J=7.4, 1.3 Hz, 1H), 6.98-6.89 (m, 3H), 6.78 (brs, 1H), 4.16-4.04 (m, 2H), 4.00 (t, J=6.2 Hz, 2H), 3.87-3.35 (m, 8H), 2.70 (s, 2H), 1.82-1.64 (m, 5H), 1.45 (s, 9H), 1.21-1.11 (m, 2H).

### Step 4: N-phenyl-4-(3-(2-(piperidin-4-yl)ethoxy)benzoyl)piperazine-1-carboxamide (S18)

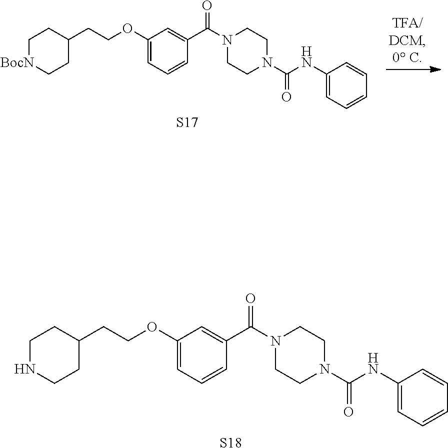 [SMILES: CC(C)(C)OC(=O)N1CCC(CCOc2cccc(C(=O)N3CCN(C(=O)Nc4ccccc4)CC3)c2)CC1 ; "S17" ; O=C(Nc1ccccc1)N1CCN(C(=O)c2cccc(OCCC3CCNCC3)c2)CC1 ; "S18"]

S18 was prepared as described for S6 from S17 (175 mg, 0.33 mmol) and was used directly without further purification.

### Step 5: 4-(3-(2-(1-acetylpiperidin-4-yl)ethoxy)benzoyl)-N-phenylpiperazine-1-carboxamide (TM98)

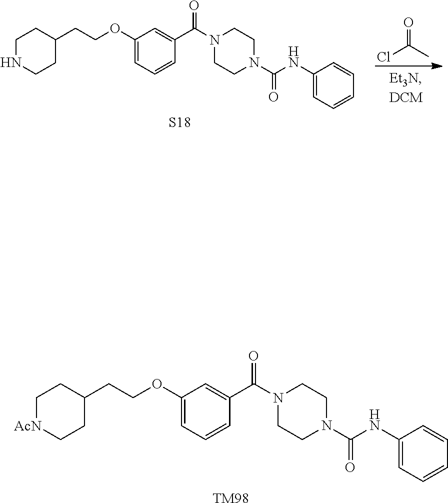

S18 (25 mg, 0.0573 mmol) was dissolved in DCM (1.5 mL). To this solution was added Et₃N (16 μL, 0.115 mmol) and acetyl chloride (4.9 μL, 0.0688 mmol) on ice. The reaction mixture was stirred at room temperature for 2 h. After completion, the reaction was quenched with saturated NaHCO₃ and extracted with ethyl acetate. The combined organic layer was washed with brine, dried over anhydrous Na₂SO₄ and concentrated in vacuo. The crude residue was purified through silica gel chromatography to give S18 as a colorless oil (20 mg, 73%). ¹H NMR (500 MHz, Chloroform-d) δ 7.38-7.24 (m, 5H), 7.04 (t, J=7.3 Hz, 1H), 6.94 (ddt, J=10.3, 6.1, 2.5 Hz, 3H), 6.86-6.75 (m, 1H), 4.60 (d, J=13.1 Hz, 1H), 4.02 (t, J=5.9 Hz, 2H), 3.92-3.36 (m, 9H), 3.04 (t, J=13.0 Hz, 1H), 2.54 (t, J=13.0 Hz, 1H), 2.08 (s, 3H), 1.84-1.71 (m, 5H), 1.27-1.10 (m, 2H). ¹³C NMR (125 MHz, Chloroform-c) 6 170.50, 168.98, 159.17, 155.24, 138.92, 136.64, 129.92, 129.02, 123.52, 120.34, 119.17, 116.37, 113.28, 65.57, 46.77, 44.26, 41.90, 35.57, 33.20, 32.78, 31.83, 21.61. HRMS (ESI): calcd for C₂₇H₃₅N₄O₄ [M+H]*, 479.2658; found, 479.2653.

### Example 5: Synthesis of TM112

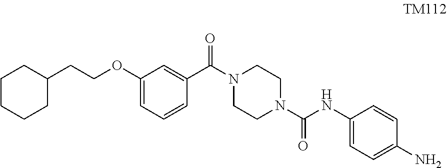 [SMILES: Nc1ccc(NC(=O)N2CCN(C(=O)c3cccc(OCCC4CCCCC4)c3)CC2)cc1]

### Step 1: N-(4-isocyanatophenyl)acetamide (S20)

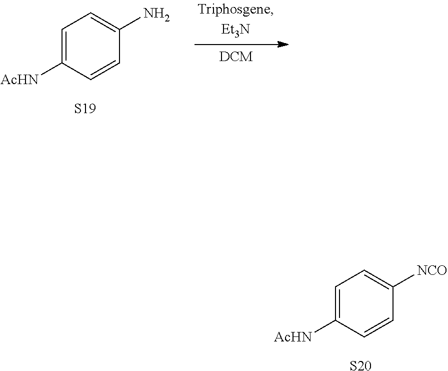

To a solution of triphosgene (311.6 mg, 1.05 mmol) in DCM (6 mL) was added a solution of Et₃N (0.9 mL, 6.45 mmol) and S19 (450.5 mg, 3 mmol) in DCM (6 mL) dropwise on ice. The mixture was continuously stirred at rt for 1h. The reaction was quenched with saturated NaHCO₃ dropwise on ice. The mixture was then diluted with water and extracted with ethyl acetate. The combined organic layer was washed with brine, dried over anhydrous Na₂SO₄ and concentrated in vacuo to give S20 which was used directly without further purification.

### Step 2: N-(4-Acetamidophenyl)-4-(3-(2-cyclohexylethoxy)benzoyl)piperazine-1-carboxamide (S21)

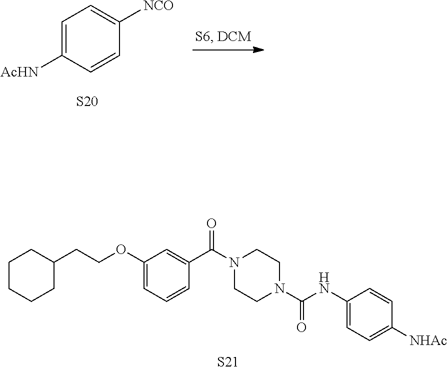

S21 was prepared as described for TM2 from S6 (120 mg, 0.376 mmol) and N-(3-isocyanatophenyl)acetamide (79.5 mg, 0.451 mmol) as a white solid (100.5 mg, 54%). ¹H NMR (500 MHz, Chloroform-d) δ 7.40 (d, J=8.4 Hz, 2H), 7.33-7.26 (m, 3H), 7.15 (brs, 1H), 6.98-6.89 (m, 3H), 6.39 (brs, 1H), 3.99 (t, J=6.7 Hz, 3H), 3.92-3.35 (m, 8H), 2.15 (s, 3H), 1.77-1.62 (m, 7H), 1.53-1.44 (m, 1H), 1.29-1.10 (m, 3H), 1.01-0.90 (m, 2H).

### Step 3: N-(4-aminophenyl)-4-(3-(2-cyclohexylethoxy)benzoyl)piperazine-1-carboxamide (TM112)

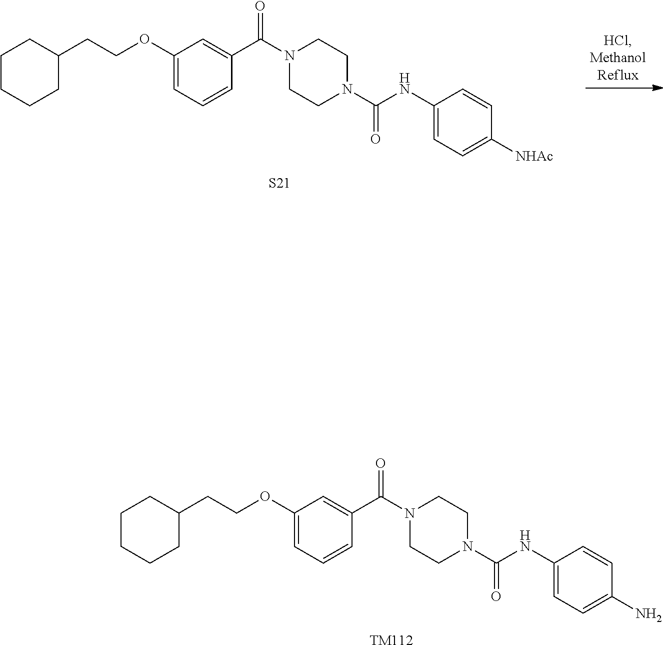

To a solution of S21 (80 mg, 0.161 mmol) in methanol (2 mL) was added 2 N HCl (4 mL).

The reaction was refluxed for 2 h. After cooling down to rt, the reaction mixture was basified with saturated NaHCO₃ on ice and extracted with ethyl acetate. The combined organic layer was washed with brine, dried over anhydrous Na₂SO₄ and concentrated in vacuo. The crude residue was purified through silica gel chromatography to give TM112 as colorless oil (23.8 mg, 33%). ¹H NMR (500 MHz, Chloroform-d) δ 7.31 (t, J=8.0 Hz, 1H), 7.09 (d, J=8.6 Hz, 2H), 6.98-6.90 (m, 3H), 6.63 (d, J=8.6 Hz, 2H), 6.23 (s, 1H), 4.00 (t, J=6.7 Hz, 2H), 3.93-3.26 (m, 10H), 1.78-1.64 (m, 7H), 1.55-1.45 (m, 1H), 1.31-1.15 (m, 3H), 0.97 (qd, J=12.1, 3.0 Hz, 2H). ¹³C NMR (125 MHz, Chloroform-d) δ 170.63, 159.47, 155.85, 143.21, 136.61, 129.85, 129.80, 123.29, 118.97, 116.49, 115.70, 113.21, 66.32, 44.26, 36.68, 34.66, 33.42, 26.64, 26.37.

### Example A. Identification of TM2 as a Novel TEAD Auto-Palmitoylation Inhibitor

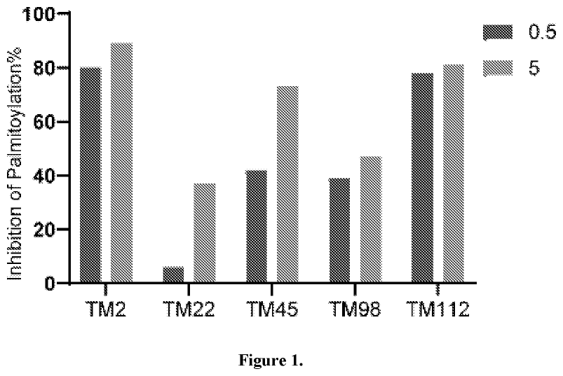

To identify new chemotypes that could inhibit TEAD auto-palmitoylation, a library containing about 30,000 non-proprietary medicinal chemistry compounds with three rounds of click-ELISA assay was screened (Lanyon-Hogg et al., 2015), through the Astellas-MGH research collaboration by using the recombinant TEAD2 and TEAD4 YBD proteins. The inhibition of ZDHHC2 was used as a selectivity filter. It was found that several hits share a common 4-(3-(2-cyclohexylethoxy)benzoyl)-piperazine-1-carboxamide moiety (data not shown, with micromolar potency in TEAD palmitoylation assays in vitro). The main variation is located at the N-substituent of the urea moiety with frequent incorporation of heteroarenes. Inspired by this structural convergence, first a series of derivatives with variable substituents at the urea moiety was designed, represented by TM2 and TM22. TEAD2 auto-palmitoylation in vitro assay was used to evaluate their potency. Compared to heteroaryl group, phenyl substituents showed stronger inhibition on TEAD2 auto-palmitoylation (TM2 vs. TM22, FIG. 1). Inspired by these results, the tolerance level was explored by increasing hydrophilicity of TM2. As illustrated by TM45 and TM98, hydrophilic groups at the left cyclohexyl ring significantly decrease the activities, while the phenyl moiety at the right-side of the urea moiety is well tolerated. Overall, TM2 was identified as the most potent compound (FIG. 1) and selected for further biological evaluations.

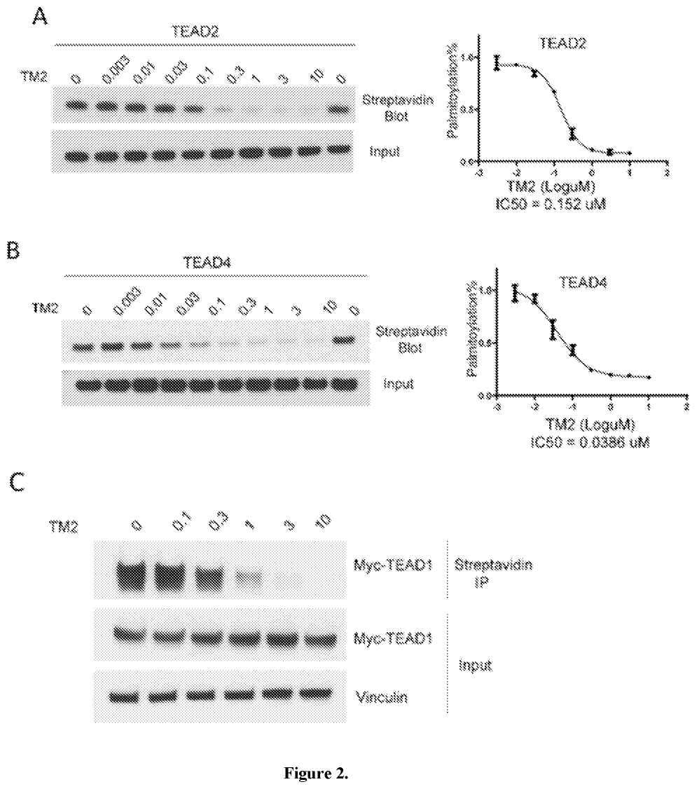

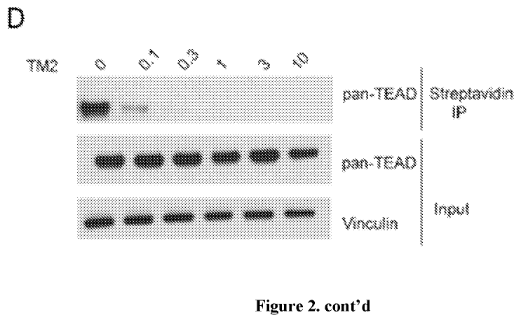

TEAD family consists of four homologous members, TEAD1-4, which share highly conserved domain architectures (Pobbati and Hong, 2013). It was found that TM2 inhibits TEAD2 palmitoylation with an IC₅₀ value of 156 nM (FIG. 2A). Encouragingly, TM2 displays an even more potent effect on TEAD4 auto-palmitoylation with an IC₅₀ of 38 nM (FIG. 2B). To study its effects on cellular TEAD palmitoylation, Myc-TEAD1 was overexpressed in HEK293A cells and treated with TM2 at different doses. As FIG. 2C shows, TM2 dramatically suppresses Myc-TEAD1 palmitoylation in cells in a dose-dependent manner. Furthermore, treatment of TM2 also significantly inhibits endogenous TEAD1-4 palmitoylation using an antibody recognizing pan-TEADs (FIG. 2D). Even at as low as 100 nM, Pan-TEAD palmitoylation was diminished. Collectively, these results suggested that TM2 is a potent and pan-inhibitor of palmitoylation of TEAD family proteins.

### Example B. Co-Crystal Structure of TEAD2 Complexed with TM2

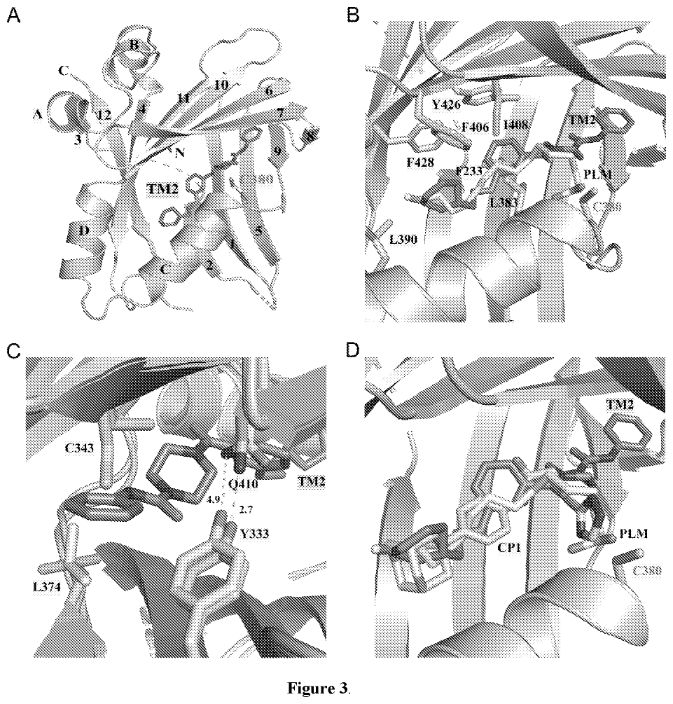

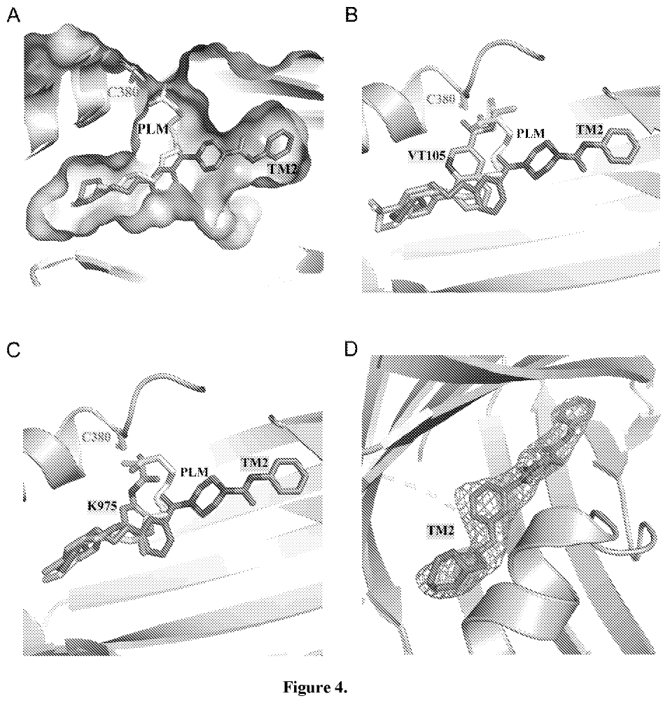

The co-crystal structure of TEAD2 YBD in complex with TM2 at 2.4 Å resolution was determined (FIGS. 3 and 4 and Table B1). As shown in FIG. 3B, the (2-cyclohexylethoxy)phenyl moiety of TM2 is surrounded by several hydrophobic residues, such as F233, L383, L390, F406, 1408, Y426, and F428, enabling strong hydrophobic interactions, which is very similar to the interaction mode of TEAD with the fatty acyl chain of palmitic acid.

However, by superposing the TEAD2-TM2 with TEAD2-PLM structures (PDB 5HGU)(Chan et al., 2016), a new feature of TM2 binding was observed (FIG. 3B). Unlike palmitic acid with its head group pointing towards residue C380, the urea moiety of TM2 exhibits a completely different orientation and sticks into a new side pocket, which has never been reported before to be involved in TEAD inhibitor binding and is only accessible by rearranging the side chains upon TM2 binding (FIG. 3B and FIG. 4A). TM2 binding drives significant conformational changes in the side chains of residues C343 and L374, which makes space for TM2 insertion (FIG. 3C). Additionally, TM2 binding causes the side chain movement in residue Q410 and Y333, which reduces the distance between the nitrogen atom of Q410 and the oxygen atom of Y333 from 4.9 Å to 2.7 Å to allow the formation of favorite electrostatic interaction (FIG. 3C).

This binding model is highly consistent with our structure-activity relationship (SAR) results in FIG. 1 that demonstrate that the left hydrophobic tail is repulsive to incorporate hydrophilicity, while the urea moiety is tolerated. The surface electrostatics of the TM2 binding pocket (FIG. 2A) also illustrated that the (2-cyclohexylethoxy)phenyl moiety inserts into a nearly neutral environment, while the urea is buried in a pocket bearing electronegative properties. Furthermore, the electronegative carbonyl, which links benzene and piperazine, is spatially adjacent to electropositive electrostatics.

We then set to figure out whether this unexpected binding model is unique to TM2, compared to other TEAD inhibitors. The co-crystal structures of TEAD YBD in complex with PLM, TM2, and other known TEAD inhibitors, including MGH-CP1 (PDB 6CDY) (Li et al., 2020a), K975 (PDB 7CMM) (Kaneda et al., 2020) and VT105 (PDB 7CNL) (Tracy T. Tang et al., 2021), were superposed (FIG. 3D and FIGS. 4B and 4C). Although PLM and these TEAD inhibitors are co-crystallized with different members of TEAD family of proteins, the highly homologous structures of TEAD YBD allowed us to compare their binding modes. Consistent with previously reported results, MGH-CP1, VT105 or K975 adopts almost the same binding mode as PLM and fits very well with the PBP. However, the scenario depicted by TM2 is quite different, which provides new insights into the structural adaptability for development of TEAD inhibitors. Considering relatively higher hydrophilicity in the new side pocket, there will be much more space to balance the lipophilicity of TEAD inhibitors and improve drug-like properties, such as solubility and metabolism (Waring, 2010).

TABLE B1
Data collection and structure refinement statistics.

| | TEAD2-TM2 | |
|---|---|---|
| Data collection | | |
| Wavelength (Å) | 0.979 | |
| Resolution (Å²) | 50.00-2.40 (2.44-2.40) | |
| Space group | C2 | |
| Unit cell dimensions | | |
| a, b, c (Å) | 124.07, 62.29, 79.91 | |
| α, β, γ (°) | 90.0, 117.7, 90.0 | |
| Redundancy | 3.6 | (2.8) |
| Completeness (%) | 97.0 | (83.7) |
| Reflections (unique) | 20, 774 | |
| I/σ_{I} | 24.1 | (1.5) |
| R_{sym} (%) | 5.2 | (68.0) |
| Rₚᵢₘ (%) | 3.1 | (45.6) |

| CC_{1/2}^{a} | 0.720 |
|---|---|
| Refinement | |
| No. of non-hydrogen atoms | 3, 393 |
| Protein | 3, 299 |
| Ligand | 64 |
| Water | 30 |
| Average B factor (Å²) | 48.9 |
| Protein | 49.0 |
| Ligand | 47.2 |
| Water | 42.7 |
| Rwork/Rfree (%) | 18.38/23.46 |
| RMSDs | |
| Bond length (Å) | 0.008 |
| Bond angle (°) | 1.099 |
| Favored/allowed/outliers (%) | 92.75/7.25/0.00 |

Values for the highest resolution shell are given in parentheses.
^{a}CC_{1/2} values shown are for the highest resolution shell.

### Example C. TM2 Inhibits TEAD-YAP Association and TEAD-YAP Transcriptional Activity

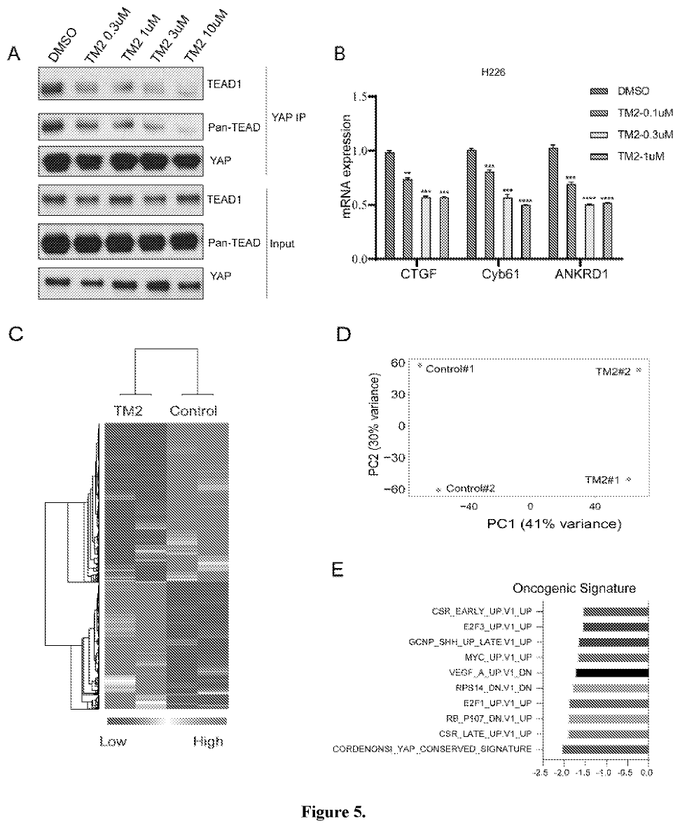

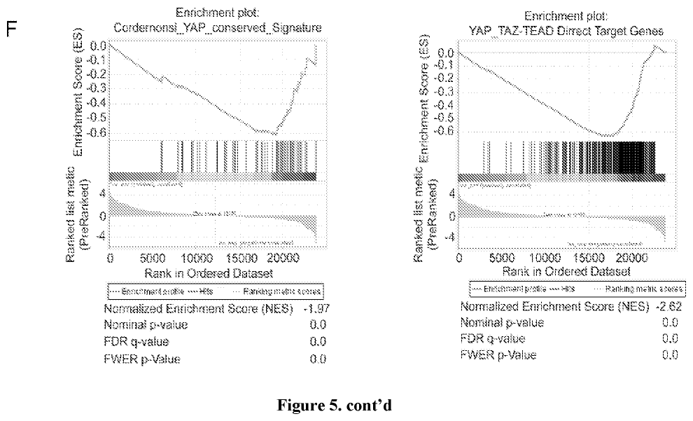

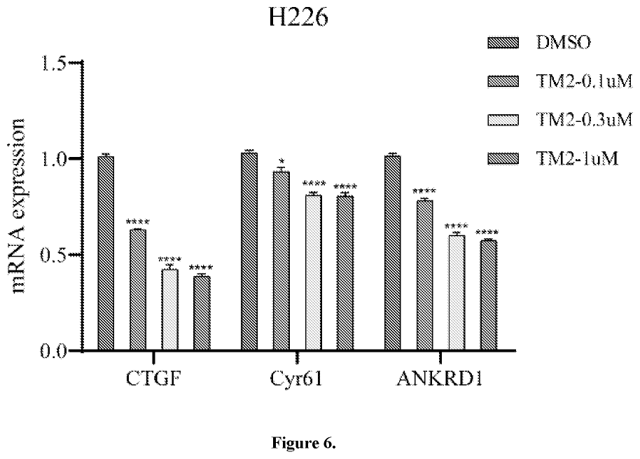

TEAD auto-palmitoylation plays important roles in regulation of TEAD-YAP interaction. To confirm whether TM2 functions through blockade of TEAD-YAP binding, TM2 was tested in a malignant pleural mesothelioma (MIPM) cell line H226 cells, which is deficient with NF2 and highly dependent on TEAD-YAP activities (Kaneda et al., 2020; Tracy T Tang et al., 2021). YAP co-immunoprecipitation (IP) experiments indicated that TM2 dramatically blocked the association of YAP with endogenous TEAD1 as well as pan-TEAD in a dose-dependent manner (FIG. 5A). Next, the effects of TM2 were evaluated in the expressions of TEAD-YAP target genes, represented by CTGF, Cyr61 and ANKDR1. After treatment of TM2, the expression level CTGF and ANKDR1 were significantly suppressed at both 24 and 48 h, while Cyr61 show strong response at 48h (FIG. 5B and FIG. 6).

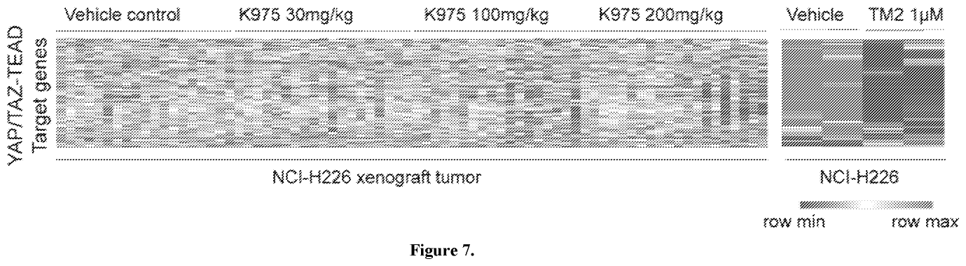

In order to systemically evaluate the effect of TM2 on YAP/TAZ-TEAD transcriptional activation, RNA-seq analysis was performed (FIG. 5C). YAP/TAZ-dependent H226 cells were treated with or without TM2. Principle component analysis (PCA) was performed, a mathematical algorithm reducing the dimensionality of the data while retaining most of the variation in the data sets. The samples were plotted and indicated the TM2 treatment substantially altered the gene sets at PC1 in H226 cells (FIG. 5D). Gene set enrichment analysis (GSEA) was performed to analyze the transcriptional signature gene sets from Molecular Signature Database. It showed that YAP signature was the top enriched signature according to the Normalized Enrichment Score (NES) (FIG. 5E). To further validate the effects of TM2 on YAP/TAZ signaling, the Cordernonsi_YAP_conserved_Signature and YAP_TAZ-TEAD Direct Target Genes were determined (Zanconato et al., 2015). Consistently, YAP/TAZ signature was significantly enriched in downregulation phenotype in both of gene sets (FIG. 5F). The specificity of TM2 was then compared with that of irreversible TEAD inhibitor K975 that showed strong antitumor effects in H226 xenograft tumor. Through global analysis of YAP/TAZ-TEAD direct target genes in H226 xenograft tumor treated with three doses of K975 (p.o.) and H226 cells treated with 1 μM TM2 (Kaneda et al., 2020; Zanconato et al., 2015), it was found that TM2 was more efficient to block YAP/TAZ-TEAD target genes relative to K975 in H226 xenograft tumors (FIG. 7), highlighting the high specificity of our reversible inhibitors. Taken together, TM2 was identified as a potent disruptor that can specifically attenuate outputs of Hippo pathway.

### Example D. TM2 Inhibits YAP-Dependent Organoids Growth and Cancer Cell Proliferation

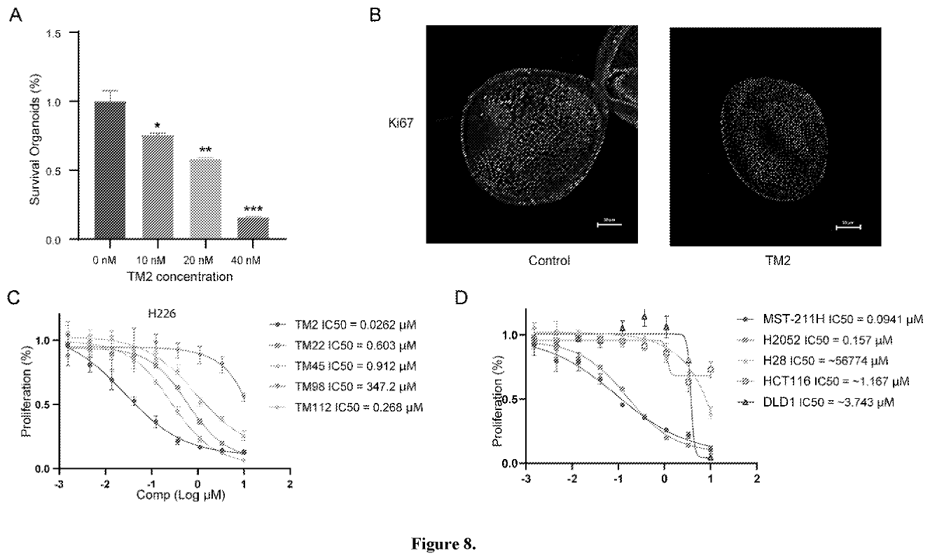

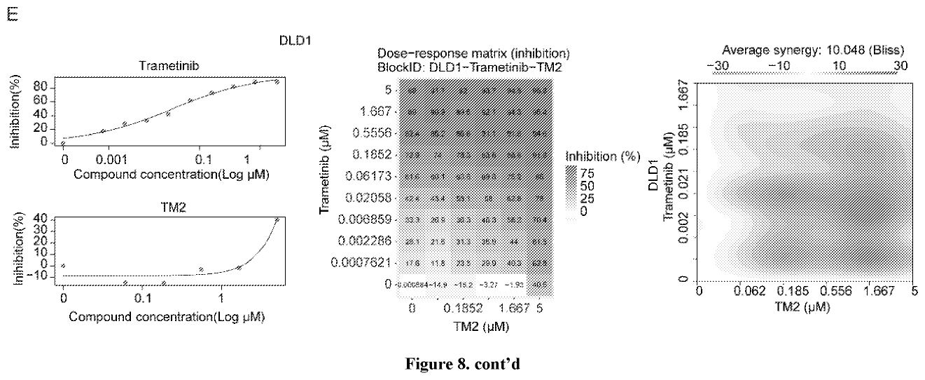

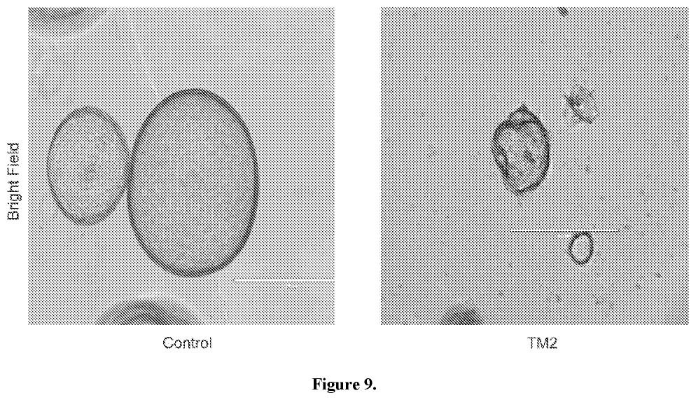

YAP activity has been shown to be critical for the growth of liver organoid (Planas-Paz et al., 2019). Therefore, mouse hepatic progenitor ex vivo organoids were used to further investigate the effects of TM2 in a physiologically relevant model. As shown in FIG. 8A, TM2 impaired the sustainability of organoids growth in a dose dependent manner, with more than 85% of disruption at 40 nM. Consistently, Ki67 positive cells for organoids maintenance in 3D culture were significantly diminished upon TM2 treatment (FIG. 8B and FIG. 9).

Pleural mesothelioma (MPM) is a type of aggressive tumor, associated with exposure to asbestos fibers (Rossini et al., 2018). Despite several standard therapies, such as surgery, radiotherapy, chemotherapy and immunotherapies, MPM patients still suffer poor prognosis with a median survival of only 8-14 months (Nicolini et al., 2020). NF2 and LATS2, the upstream components of Hippo pathway, are frequently observed to be inactivated in malignant mesothelioma (MM), leading YAP activation in more than 70% of analyzed primary MM tissues (Murakami et al., 2011; Sekido, 2018). Therefore, MM would be a good model to study the therapeutic effects of TM2 on Hippo signaling defective cancers. Encouraged by the strong inhibition of TEAD-YAP transcriptional activities in H226 cells, anti-proliferative activities of TM2 in this cell line were first evaluated. As shown in FIG. 8C, H226 cells exhibited striking vulnerability to TM2 treatment with an IC₅₀ value of 26 nM, consistent with its potency in blocking TEAD palmitoylation in vitro and in cells. Other derivatives, including TM22, TM45, TM98, TM112 were less potent than TM2, which correlated well with their in vitro activities (FIG. 8C). In addition, the effects of TM2 in two other MPM cell lines were also studied, MSTO-211H and NCI-H2052, which harbors Lats1/2 deletion/mutations, and NF2-deficiency, respectively (Kaneda et al., 2020; Lin et al., 2017; Miyanaga et al., 2015). Consistently, TM2 also significantly inhibited cell proliferation of MSTO-211H and NCI-H2052 cells (FIG. 8D) with IC₅₀ values of 94 nM and 157 nM, respectively. In comparison, TM2 shows no significant inhibition in the Hippo WT mesothelioma cells, NCI-H28 with IC₅₀>5 μM (Tanaka et al., 2013) (FIG. 8D, suggesting TM2 is specific to YAP-activated cancer cells.

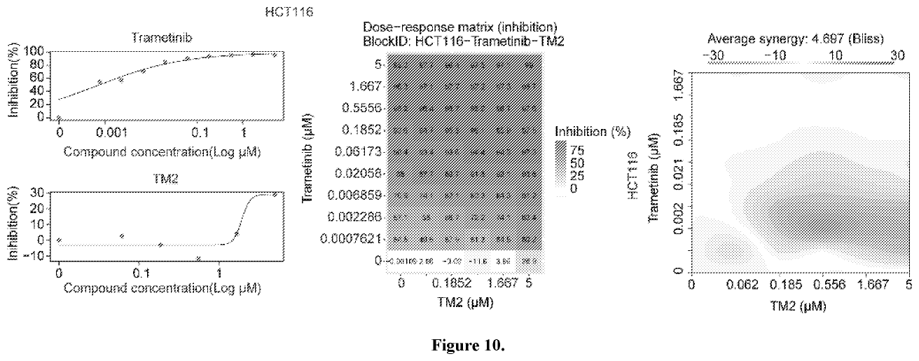

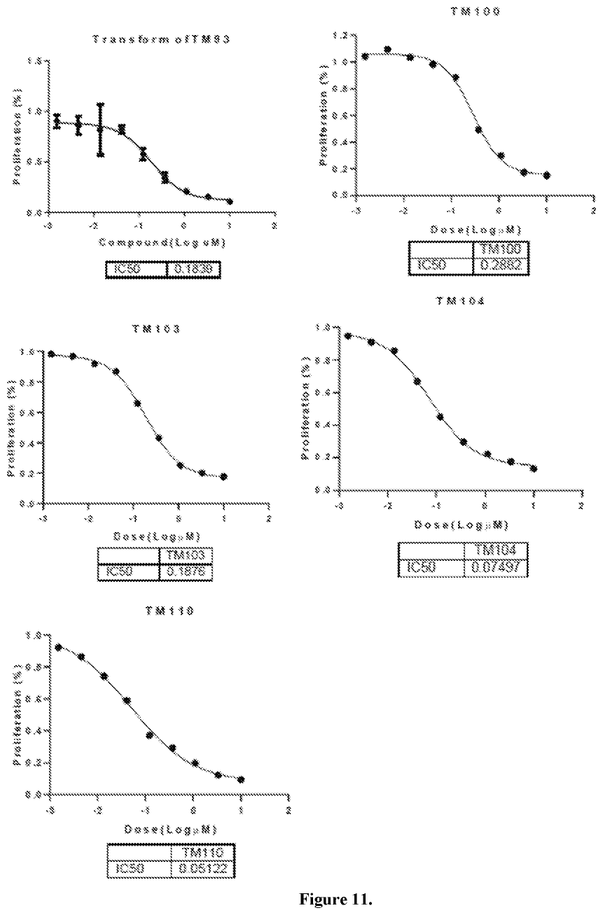

Currently, TEAD inhibitors mainly show promising therapeutic potentials in mesothelioma, with limited activities in other YAP-dependent cancer cells. Given that deregulated Hippo signaling is implicated in many human cancers (Harvey et al., 2013), it is important to test the efficacy of TEAD inhibitors in cancers beyond mesothelioma, which will deepen our understanding of therapeutic spectrum of blocking TEAD-YAP activities. Therefore, TM2 in colorectal cancer (CRC) was evaluated, as Hippo pathway has been shown to regulate the progression of CRC (Della Chiara et al., 2021; Jin et al., 2021; Pan et al., 2018). However, TM2 did not exhibit strong inhibition on cell proliferation of two CRC cell line (FIG. 8D), HCT116 and DLD1. These results suggested suppression of Hippo transcriptional activities in CRC alone might not be sufficient to inhibit cell growth, as observed in mesothelioma. Indeed, YAP are found to be capable of rescuing cell viability in HCT116 with loss function of KRAS, implying KRAS signaling might also account for lack of potency of TM2 in CRC. Hence, a drug combination matrix analysis across 5 doses of TM2 and 9 doses of MEK inhibitor trametinib in HCT116 and DLD1, respectively, were performed. Encouragingly, strong inhibitory effects and substantial synergy in both of two cell lines were observed (FIG. 8E and FIG. 10), suggesting that combining TEAD inhibitors with other therapies might be a good strategy to broaden their therapeutic applications in near future. Together, our data highlights that TM2 might have appealing potentials to antagonize carcinogenesis driven by aberrant YAP activities.

### Example E. TM2 Analogues with Improved Metabolic Stability

Metabolic stability tests were done through Scripps Florida DMPK Core. TM93 and TM100 showed improved metabolic stability (Table E1 and E2).

TABLE E1
Half-life in 1 mg/mL hepatic microsomes

Species (t_{1/2} in minutes)

| Compound ID | Human | Mouse |
|---|---|---|
| sunitinib | 30.1 | 10.9 |
| TM97 | | 1.7 |
| TM93 | 11.1 | 16.7 |
| TM45 | | 11.2 |
| TM37 | 3.8 | 5.4 |
| TM100 | 5.8 | 9.6 |

TABLE E2
Intrinsic Clearance

Species (Clᵢₙₜ -μl/min/mg)

| Compound ID | Human | Mouse |
|---|---|---|
| sunitinib | 23 | 64 |
| TM97 | | 415 |
| TM93 | 62 | 41 |
| TM45 | | 62 |
| TM37 | 182 | 129 |
| TM100 | 120 | 72 |

### Example F. SAR Around TM Series Compounds

CP-718 is inactive in primary assay, however moderately stable in MLM. CP-715 show low clearance, high volume of distribution and low oral bioavailability ˜5%. CP-716 is active in cellular assays, however MLM stability is not improved. CP-717 is inactive in primary assay & has poor MLM stability.

TABLE F1

| Structure | TM-2 | 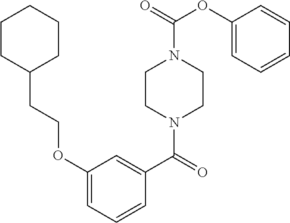 [SMILES: O=C(Oc1ccccc1)N1CCN(C(=O)c2cccc(OCCC3CCCCC3)c2)CC1] | 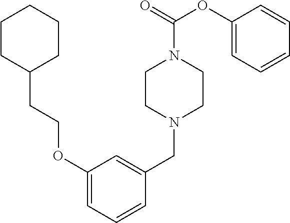 [SMILES: O=C(Oc1ccccc1)N1CCN(Cc2cccc(OCCC3CCCCC3)c2)CC1] |
|---|---|---|---|
| Compound code | MGH-CP- | MGH-CP-714 | MGH-CP-718 |
| | 685 | | |
| MW, PSA, clogP | 435.6, 62, | 436.5, 59, | 422.6, 42, |
| | 4.6 | 5.91 | 6.0 |
| WB:% inh. @ | 0.344 | 16/94 | −4/11 |
| 1/10 μM | | | |
| DHODH(H):(GI50 | 5/6/15 | | |
| μM/1,10 μM) | | | |
| H226:(GI50 | 0.073 | 2.643 | IP |
| μM/1,10 μM) | | | |
| KNS62:(GI50 | 37/28 | 10/48 | IP |
| μM/1,10 μM) | | | |
| Aq Sol: (μM) | 2.3 | <2 | IP |
| Met stab MLM; | 36/4(M); | 29/<5 | 59/13 |
| HLM(15/60 min.) | 44/6/(H) | | |
| | | | 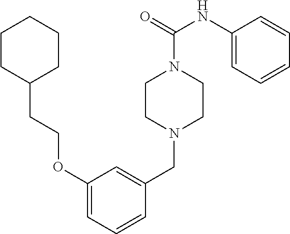 [SMILES: O=C(Nc1ccccc1)N1CCN(Cc2cccc(OCCC3CCCCC3)c2)CC1] |
| | | Compound code | MGH-CP-715 |
| | | MW, PSA, clogP | 421.6, 45, |
| | | | 6.2 |
| | | WB:% inh. @ | 35/95 |
| | | 1/10 μM | |
| | | DHODH(H):(GI50 | |
| | | μM/1,10 μM) | |
| | | H226:(GI50 | 16/69 |
| | | μM/1,10 μM) | |
| | | KNS62:(GI50 | 4/57 |
| | | μM/1,10 μM) | |
| | | Aq Sol: (μM) | <2 |
| | | Met stab MLM; | 66/15 |
| | | HLM(15/60 min.) | |

TABLE F2A

| CP-715 | t_{1/2}, β | C₀/Cₘₐₓ | Tₘₐₓ | AUC₍₀₋ₜ₎ | AUC_{(0-inf)} |
|---|---|---|---|---|---|
| PK | (h) | (ng/mL) | (h) | (h*ng/mL) | (h*ng/mL) |
| 1 mg/Kg (IV) | 3.71 | 834 | — | 663 | 736 |
| 10 mg/Kg (PO) | 1.87 | 108 | 0.25 | 394 | 422 |

TABLE F2B

| | CL | V_{SS} | MRT | F |
|---|---|---|---|---|
| CP-715 PK | (mL/min/kg | (L/kg) | (h) | (%) |
| 1 mg/Kg (IV) | 22.6 | 3.61 | 1.47 | ~5.7 |
| 10 mg/Kg (PO) | — | — | 2.62 | |

TABLE F3A

| Structure | TM-2 | 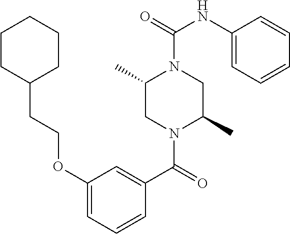 | 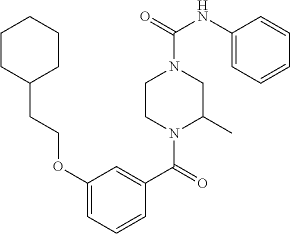 |
|---|---|---|---|
| Compound | MGH-CP- | MGH-CP-709 | MGH-CP-710 |
| code | 685 | | |
| MW, PSA, | 435.6, 62, | 463.6, 62, 6.4 | 449.6, 62, 5.89 |
| clogP | 4.6 | | |
| WB:% inh. | 0.344 | 45/77 | 54/81 |
| @1/10 μM | | | |
| H226:(GI50 | 0.073 | 1.974 | 1.04 |
| μM/1,10 μM) | | | |
| KNS62:(GI50 | 37/28 | 28/46 | 35/51 |
| μM/1,10 μM) | | | |
| HCC95:(GI50 | 37/65 | | |
| μM/1,10 μM) | | | |
| Aq Sol: (μM) | 2.3 | 2 | <2 |
| Met stab | 36/4(M); | 26/<5 | 41/<5 |
| MLM; | 44/6/(H) | | |
| HLM(15/60 | | | |
| min.) | | | |

TABLE F3A

| Structure | TM-2 | 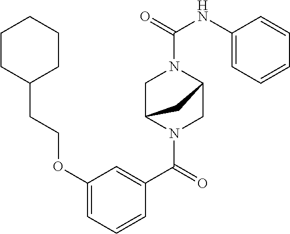 [SMILES: O=C(Nc1ccccc1)N1C[C@@H]2C[C@H]1CN2C(=O)c1cccc(OCCC2CCCCC2)c1] | 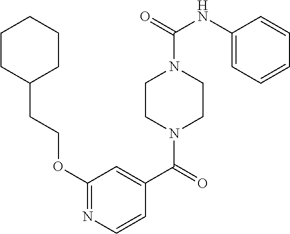 [SMILES: O=C(Nc1ccccc1)N1CCN(C(=O)c2ccnc(OCCC3CCCCC3)c2)CC1] |
|---|---|---|---|
| Compound | MGH-CP- | MGH-CP-717 | MGH-CP-716 |
| code | 685 | | |
| MW, PSA, | 435.6, 62, | 447.6, 62, 5.5 | 436.6, 74, 4.8 |
| clogP | 4.6 | | |
| WB:% inh. | 0.344 | −2/22 | 59/96 |
| @1/10 μM | | | |
| H226:(GI50 | 0.073 | IP | 0.236 |
| μM/1,10 μM) | | | |
| KNS62:(GI50 | 37/28 | IP | 54/60 |
| μM/1,10 μM) | | | |
| HCC95:(GI50 | 37/65 | | |
| μM/1,10 μM) | | | |
| Aq Sol: (μM) | 2.3 | IP | IP |
| Met stab | 36/4(M); | 6/<5 | 40/<5 |
| MLM; | 44/6/(H) | | |
| HLM(15/60 | | | |
| min.) | | | |

### Example G. Oral Bioavailability of MGH-CP-715 in Male CD-1Mice

TABLE G1A

| | t_{1/2}, β | C₀/Cₘₐₓ | Tₘₐₓ | AUC₍₀₋ₜ₎ | AUC_{(0-inf)} |
|---|---|---|---|---|---|
| Parameters | (h) | (ng/mL) | (h) | (h*ng/mL) | (hr*ng/mL) |
| 1 mg/Kg (IV) | 3.71 | 834 | — | 663 | 736 |
| 10 mg/Kg(PO) | 1.87 | 108 | 0.25 | 394 | 422 |

TABLE G1B

| | CL | V_{SS} | MRT | F |
|---|---|---|---|---|
| Parameters | (mL/min/kg) | (L/kg) | (h) | (%) |
| 1 mg/Kg (IV) | 22.6 | 3.61 | 1.47 | ~5.7 |
| 10 mg/Kg(PO) | — | — | 2.62 | |

TABLE G2

| | % Parent |
|---|---|
| | remained (15/60 min) |
| Compounds | MLM |
| MGH-CP-715 | 66/15 |

### Example H. Metabolic Stability

Metabolic stability significantly correlates with hydrophilicity. Carbamate type tends to be more stable than urea type.

TABLE H1

| | CLint(mL/min/kg) | |
|---|---|---|
| Urea type | human/mouse | logD 7.4 |
| TM-2 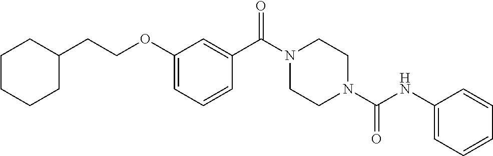 [SMILES: O=C(Nc1ccccc1)N1CCN(C(=O)c2cccc(OCCC3CCCCC3)c2)CC1] | not tested | 4.60 |
| TM-23 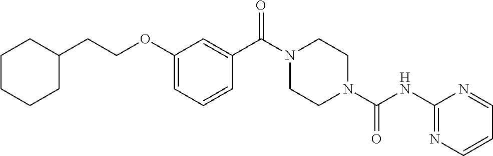 [SMILES: O=C(Nc1ncccn1)N1CCN(C(=O)c2cccc(OCCC3CCCCC3)c2)CC1] | 109/156 | 2.38 |

TABLE H2

| | CLint | |
|---|---|---|
| | (mL/min/kg) | |
| Carbamate type | human/mouse | logD 7.4 |
| 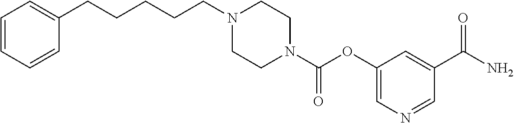 [SMILES: NC(=O)c1cncc(OC(=O)N2CCN(CCCCCc3ccccc3)CC2)c1] | 66/120 | 3.80 |
| 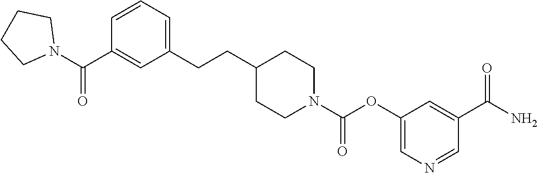 [SMILES: NC(=O)c1cncc(OC(=O)N2CCC(CCc3cccc(C(=O)N4CCCC4)c3)CC2)c1] | 86/90 | 2.60 |
| 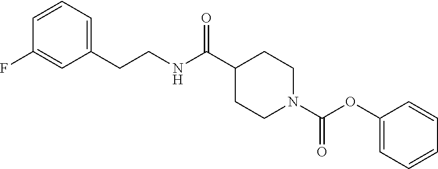 [SMILES: O=C(NCCc1cccc(F)c1)C1CCN(C(=O)Oc2ccccc2)CC1] | 54/87 | 2.59 |
| 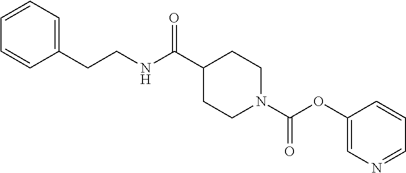 [SMILES: O=C(NCCc1ccccc1)C1CCN(C(=O)Oc2cccnc2)CC1] | 41/98 | 2.54 |
| 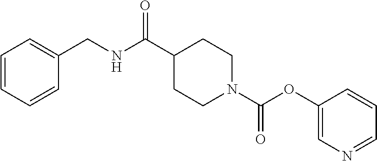 [SMILES: O=C(NCc1ccccc1)C1CCN(C(=O)Oc2cccnc2)CC1] | 24/63 | 2.50 |
| 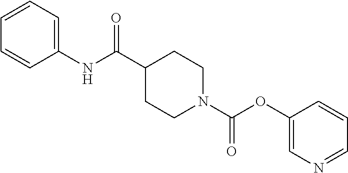 [SMILES: O=C(Nc1ccccc1)C1CCN(C(=O)Oc2cccnc2)CC1] | no depletion (stable)/ 47 | 2.16 |

What is claimed is:

1. A compound of Formula (I) 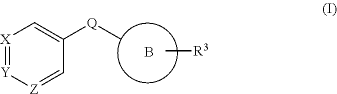
or a pharmaceutically acceptable salt thereof, wherein:
X is —-R¹, CH, N, or CR²;
Y is —CA-R¹, CH, N, or CR²;
Z is —CA-R¹, CH, N, or CR², wherein only one of X, Y, and Z is —CA-R¹;
Q is C(═O), S(═O), S(O₂), 4-5 membered spiroheterocyclyl, C1-C6 alkylene, or a bond;
A is O or NH;
R¹ is C1-C6 alkyl optionally substituted with:
(v) C3-C8 cycloalkyl optionally substituted with 1-2 substituents independently selected from halogen and C1-C6 alkyl,
(vi) 4-10 membered heterocyclyl optionally substituted with C1-C6 alkyl, acyl, or a C-linked ester,
(vii) 5-6 membered heteroaryl optionally substituted with C1-C6 alkyl or C1-C6 haloalkyl, or
(viii) phenyl optionally substituted with C1-C6 alkyl or C1-C6 haloalkyl;
each R² is independently halogen, cyano, C1-C6 alkyl, C1-C6 alkoxy, or C1-C6 haloalkyl;
Ring B is phenyl optionally substituted with 1-2 independently selected C1-C6 alkyl, 5-6 membered heteroaryl optionally substituted with 1-2 independently selected C1-C6 alkyl, or 4-10 membered heterocyclyl optionally substituted with 1-2 independently selected C1-C6 alkyl;
R³ is —C(═O)NR^{A}, —C(═O)OR^{A}, —NH(C═O)R^{A}, —NHC(═O)NH^{A}, or —C1-C6 alkyl(NHC(═O)NH)R^{A};
R^{A} is phenyl, 5-6 membered heterocyclyl, or 5-6 membered heteroaryl, each optionally substituted with 1-2 independently selected R^{A1};
each R^{A1} is independently —NR^{B}R^{C}, C-linked ester, —CO₂H, —S(O₂)NH₂, —NHC(═O)C1-C6 alkyl, or C1-C6 alkyl optionally substituted with hydroxyl, wherein
R^{B} and R^{C} is independently hydrogen, C1-C6 alkyl, C1-C6 haloalkyl, or
R^{B} and R^{C}, together with the nitrogen to which they are attached form a 4-6 membered heterocyclyl optionally substituted with 1-2 independently selected halogen, C1-C6 alkyl, C1-R⁶ haloalkyl, hydroxyl, or amino.

2. The compound of claim 1, wherein X is —CA-R¹.

3. The compound of claim 1, wherein Y is —CA-R¹.

4. The compound of claim 1, wherein Z is —CA-R¹.

5. The compound of any one of claims 1-4, wherein two of X,Y, and Z are both CH.

6. The compound of any one of claims 1-4, wherein two of X,Y, and Z are both N.

7. The compound of any one of claims 1-4, wherein two of X,Y, and Z are both CR².

8. The compound of any one of claims 1-4, wherein two of X,Y, and Z is independently CH or N; wherein one of X,Y, and Z is CH.

9. The compound of any one of claims 1-4, wherein two of X,Y, and Z is independently CH or CR²; wherein one of X,Y, and Z is CH.

10. The compound of any one of claims 1-4, wherein two of X,Y, and Z is independently N or CR²; wherein one of X,Y, and Z is N.

11. The compound of any one of claims 1-10, wherein Q is C(═O).

12. The compound of any one of claims 1-10, wherein Q is S(═O).

13. The compound of any one of claims 1-10, wherein Q is S(O₂).

14. The compound of any one of claims 1-10, wherein Q is 4-5 membered spiroheterocyclyl.

15. The compound of any one of claims 1-10, wherein Q is C1-C6 alkylene.

16. The compound of any one of claims 1-10, wherein Q is a bond.

17. The compound of any one of claims 1-16, wherein A is O.

18. The compound of any one of claims 1-16, wherein A is NH.

19. The compound of any one of claims 1-18, wherein R¹ is C1-C6 alkyl optionally substituted with C3-C8 cycloalkyl optionally substituted with 1-2 substituents independently selected from halogen and C1-C6 alkyl.

20. The compound of any one of claims 1-18, wherein R¹ is C1-C6 alkyl optionally substituted with 4-10 membered heterocyclyl optionally substituted with C1-C6 alkyl, acyl, or a C-linked ester.

21. The compound of any one of claims 1-18, wherein R¹ is C1-C6 alkyl optionally substituted with 5-6 membered heteroaryl optionally substituted with C1-C6 alkyl or C1-C6 haloalkyl.

22. The compound of any one of claims 1-18, wherein R¹ is C1-C6 alkyl optionally substituted with phenyl optionally substituted with C1-C6 alkyl or C1-C6 haloalkyl.

23. The compound of any one of claims 1-22, wherein each R² is independently halogen, cyano, C1-C6 alkyl, C1-C6 alkoxy, or C1-C6 haloalkyl.

24. The compound of any one of claims 1-23, wherein Ring B is phenyl optionally substituted with 1-2 independently selected C1-C6 alkyl.

25. The compound of any one of claims 1-23, wherein Ring B is 5-6 membered heteroaryl optionally substituted with 1-2 independently selected C1-C6 alkyl.

26. The compound of any one of claims 1-23, wherein Ring B is 4-10 membered heterocyclyl optionally substituted with 1-2 independently selected C1-C6 alkyl.

27. The compound of any one of claims 1-26, wherein R³ is —C(═O)NHR^{A}.

28. The compound of any one of claims 1-26, wherein R³ is —C(═O)OR^{A}.

29. The compound of any one of claims 1-26, wherein R³ is —NH(C═O)R^{A}.

30. The compound of any one of claims 1-26, wherein R³ is —NHC(═O)NHR^{A}.

31. The compound of any one of claims 1-26, wherein R³ is C1-C6 alkyl(NHC(═O)NH)R^{A}.

32. The compound of any one of claims 1-31, wherein R^{A} is phenyl optionally substituted with 1-2 independently selected R^{A1}.

33. The compound of any one of claims 1-31, wherein R^{A} is 5-6 membered heterocyclyl optionally substituted with 1-2 independently selected R^{A1}.

34. The compound of any one of claims 1-31, wherein R^{A} is 5-6 membered heteroaryl optionally substituted with 1-2 independently selected R^{A1}.

35. The compound of any one of claims 1-34, wherein each R^{A1} is independently —NR^{B}R^{C}, C-linked ester, —CO₂H, —S(O₂)NH₂, —NHC(═O)C1-C6 alkyl, or C1-C6 alkyl optionally substituted with hydroxyl.

36. The compound of any one of claims 1-35, wherein R^{B} and R^{C} is independently hydrogen, C1-C6 alkyl, C1-C6 haloalkyl, or
R^{B} and R^{C}, together with the nitrogen to which they are attached form a 4-6 membered heterocyclyl optionally substituted with 1-2 independently selected halogen, C1-C6 alkyl, C1-C6 haloalkyl, hydroxyl, or amino.

37. The compound of claim 1, wherein the compound of Formula (I) is Formula (I-a) 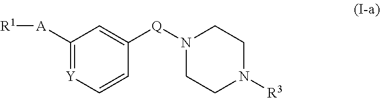
or a pharmaceutically acceptable salt thereof, wherein:
Y is CH or N; and
Q is C(═O), S(═O), S(O₂), 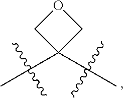 [SMILES: *C1(*)COC1]
or methylene.

38. The compound of claim 1, wherein the compound of Formula (I) is Formula (I-b) 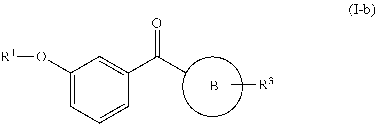
or a pharmaceutically acceptable salt thereof.

39. The compound of claim 1, wherein the compound of Formula (I) is Formula (I-c) 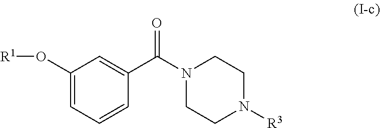
or a pharmaceutically acceptable salt thereof.

40. A composition comprising a compound of any one of claims 1-39, or a pharmaceutically acceptable salt thereof and at least one pharmaceutically acceptable excipient.

41. The composition of claim 40, wherein the compound of any one of claims 1-39, or a pharmaceutically acceptable salt thereof is a small molecule inhibitor of TEAD-YAP.

42. A method of treating cancer in a subject in need thereof, comprising administering to the subject an effective amount of a compound of any one of claims 1-39, or a pharmaceutically acceptable salt thereof, or a pharmaceutical composition of claim 40.

43. The method of claim 42, wherein the cancer is medulloblastoma, cutaneous squamous cell carcinoma, lung cancer, pancreatic cancer, esophageal cancer, liver cancer, or colon cancer.

44. The method of claim 42 or 43, wherein the cancer is medulloblastoma,

45. The method of claim 42 or 43, wherein the cancer is cutaneous squamous cell carcinoma.

46. The method of claim 42 or 43, wherein the cancer is esophageal cancer.

47. The method of claim 42 or 43, wherein the cancer is lung cancer, pancreatic cancer, melanoma, liver cancer, or colon cancer.

48. The method of claim 42 or 47, wherein the cancer is lung cancer.

49. The method of claim 42 or 47, wherein the cancer is pancreatic cancer.

50. The method of claim 42 or 47, wherein the cancer is melanoma.

51. The method of claim 42 or 47, wherein the cancer is liver cancer.

52. The method of claim 42 or 47, wherein the cancer is colon cancer.

53. The method of any one of claims 42-52, wherein the compound of any one of claims 1-39, or a pharmaceutically acceptable salt thereof, inhibits PDL1 expression and function as immune checkpoint blockade.
